(19) United States
(12) Patent Application Publication
(10) Pub. No.: US 2013/0059113 A1
(43) Pub. Date: Mar. 7, 2013

(54) Structures For Preventing Microorganism Attachment

(71) Applicants: Benjamin Hatton, Cambridge, MA (US); Joanna Aizenberg, Boston, MA (US)

(72) Inventors: Benjamin Hatton, Cambridge, MA (US); Joanna Aizenberg, Boston, MA (US)

(73) Assignee: PRESIDENT AND FELLOWS OF HARVARD COLLEGE, Cambridge, MA (US)

(21) Appl. No.: 13/575,688

(22) PCT Filed: Jan. 26, 2011
(86) PCT No.: PCT/US11/22620

§ 371 Date: Nov. 19, 2012

Related U.S. Application Data
(60) Provisional application No. 61/299,214, filed on Jan. 28, 2010.
(60) Provisional application No. 61/365,615, filed on Jul. 19, 2010.

(51) Int. Cl. A61L 29/14; B32B 3/12; B32B 3/30

(52) U.S. Cl. 428/116; 428/156; 428/158; 134/42

(57) ABSTRACT

Methods of making and using substrates having raised structures to inhibit adhesion of microorganisms are described.

### RELATED APPLICATIONS

The present application claims priority to U.S. Patent Application No. 61/299,214, filed Jan. 28, 2010 and U.S. Patent Application No. 61/365,615, filed Jul. 19, 2010, the entire contents of which are hereby incorporated by reference.

### BACKGROUND OF THE INVENTION

Contamination of surfaces by microbial attachment occurs very easily, and is the first step towards the development of bacterial biofilms as multicellular communal superorganisms (O'Toole et al., Annu. Rev. Microbiol. 54, 49-79 (2000); De Beer et al., Prokaryotes 1, 904-937 (2006); O'Toole, J. Bacteriology 185, 2687-2689 (2003)). An important consequence of bacterial contamination and population of surfaces is the infection of surgical instruments, biomedical materials and prosthetics such as catheters (Christensen et al., J. Clin. Microbiol. 22, 996-1006 (1985); Costerton et al., Ann. Rev. Microbiol. 41, 435-464 (1987); Gristina, Science 237, 1588-1595 (1987); Everaert et al, Colloids and Surfaces B: Biointerfaces 10, 179-190 (1998); Jacques et al., Microbial Ecology 13, 173-191 (1987); Hall et al., Public Health Records 79, 1021-1024 (1964); Druskin et al., J. Am. Med. Assoc. 185, 966-968 (1963); Bentley et al., J. Am. Med. Assoc. 206, 1749-1752 (1968); Corso et al., J. Am. Med. Assoc. 210, 2075-2077 (1969); Irwin et al., Yale J. Biol. Med. 46, 85-93 (1973); Michel et al., Am. J. Surgery 137, 745-748 (1979); and Shinozaki et al, J. Am. Med. Assoc. 249, 223-225 (1983)). Bloodstream infection caused by surgical instrument-, catheter- and implant-related bacterial contamination is a frequent serious complication associated with procedures involving catheters and implants (Christensen et al., J. Clin. Microbiol. 22, 996-1006 (1985); Costerton et al., Ann. Rev. Microbiol. 41, 435-464 (1987); Gristina, Science 237, 1588-1595 (1987); Everaert et al, Colloids and Surfaces B: Biointerfaces 10, 179-190 (1998); Jacques et al., Microbial Ecology 13, 173-191 (1987); Hall et al., Public Health Records 79, 1021-1024 (1964); Druskin et al., J. Am. Med. Assoc. 185, 966-968 (1963); Bentley et al., J. Am. Med. Assoc. 206, 1749-1752 (1968); Corso et al., J. Am. Med. Assoc. 210, 2075-2077 (1969); Irwin et al., Yale J. Biol. Med. 46, 85-93 (1973); Michel et al., Am. J. Surgery 137, 745-748 (1979); and Shinozaki et al, J. Am. Med. Assoc. 249, 223-225 (1983)).

Bacteria can physically attach to a vast variety of surfaces, from hydrophilic to hydrophobic, by a variety of mechanisms (O'Toole et al., Annu. Rev. Microbiol. 54, 49-79 (2000); De Beer et al., Prokaryotes 1, 904-937 (2006); O'Toole, J. Bacteriology 185, 2687-2689 (2003); Christensen et al., J. Clin. Microbiol. 22, 996-1006 (1985); Costerton et al., Ann. Rev. Microbiol. 41, 435-464 (1987); Gristina, Science 237, 1588-1595 (1987); Everaert et al, Colloids and Surfaces B: Biointerfaces 10, 179-190 (1998); Jacques et al., Microbial Ecology 13, 173-191 (1987)). The typical mechanisms include an initial deposition of proteins, known as conditioning layer, by physical or chemical adsorption, which precedes the attachment of the bacteria itself. Conditioning films, which may contain fibronectin, fibrinogen, collagen, and other proteins, coat a biomaterial surface almost immediately and provide receptor sites for bacterial or tissue adhesion (Gristina, Science 237, 1588-1595 (1987)).

The roles of these various macromolecules differs for different bacterial species. For example, Staphylococcus aureus has specific binding sites for collagen and fibronectin (Gristina, Science 237, 1588-1595 (1987)). Bacteria (or tissue cells, such as bone, endothelial cells, or fibroblasts) that approach a biomaterial surface first encounter the glycoprotenacious conditioning layer.

Surgical instruments and intravascular devices (IVD) such as catheters have many potential sources for infection. The adherence of microorganisms to the catheter surface is among the most important characteristics associated with the pathogenesis of infection. Even a single bacterium cell that successfully adheres to the surface can develop into a robust and infectious bacterial film and cause disease. Therefore an effective strategy for prevention of bacterial adhesion has been to develop surface materials that are intrinsically resistant to colonization. Various approaches have been made to coating the catheter surface with a nontoxic antiseptic or antimicrobial drug, or to incorporate such a substance into the catheter material itself (Crnich et al., Clinical Infectious Diseases 34, 1232-1242 (2002)). These anti-bacterial surfaces have been based on the principle of incorporating compounds such as Ag-particle composite structures, antiseptics, and antibiotics.

### SUMMARY OF THE INVENTION

Raised structures, and methods of using such structures to prevent, inhibit, or reduce the attachment of microorganisms onto substrates, are described. Such raised structures prevent, inhibit, or reduce the attachment of microorganisms on substrates when contacted with contaminated liquids containing a microorganism. The contact can be static due to simple exposure to a contaminated liquid or dynamic, such as contact due to splashing or pouring of the microorganism-containing liquid. Preferably, the adhesion is inhibited or reduced following temporary contact of the contaminated fluid. In certain embodiments, the contact lasts a few milliseconds to a few minutes.

In one aspect, the treated surface comprises raised superhydrophobic structures which energetically exclude bacteria, viruses, and fungi from the substrate surface by preventing wetting of the surface by a contaminated liquid under dynamic conditions (such as pouring, splashing, or sprinkling of the liquid on the surface), wherein the structures have a width, e.g., a distal width, of less than about 5 μm for bacteria and viruses, and less than about 15 μm for fungi.

In one or more embodiments, the raised structures have a width, e.g., a distal width, of less than about 2 μm.

In another aspect, the treated surface contains raised structures that physically exclude microorganisms from the substrate subsurface by providing raised structures having a interstructure spacing of less than about the length and/or transverse diameter of the microorganism contained in the contaminated liquid. The raised structures can be superhydrophobic, hydrophobic or hydrophilic.

In one or more embodiments, the treated surface contains raised structures that both energetically and physically exclude microorganisms from the substrate.

In some embodiments, the raised structures are posts. In further embodiments, the raised structures are channels. In still further embodiments, the raised structures are closed-cell structures. In still further embodiments, the raised structures are a combination of the above. The raised structures can be uniformly or regularly spaced on a base or subsurface, e.g., post arrays, regularly spaced channels and brick-like closed structures. In other embodiments, the structures are randomly spaced.

In some embodiments, the raised post structures comprise mechanically reinforced posts with cross-sections that ensure increased mechanical stability.

In some embodiments, the walls of the channel structures are not straight and comprise mechanically reinforced geometries.

In some embodiments, the raised structures comprise mechanically reinforced structures by having raised structures with basal widths greater than distal widths. In some embodiments, these raised structures comprise posts, channels or closed-cells with larger or wider bases, thereby exhibiting improved mechanical strength.

In one aspect, the substrate is coated to provide raised surfaces.

In another aspect, the raised structures are prepared as a coating on a device, such as a medical device, to prevent, inhibit, or reduce the attachment of microorganisms on to the device.

In some embodiments, the raised structures are of various shapes and dimensions (e.g., cross-section, height and width). In further embodiments, the raised structures are either isolated or interconnected. Thus, different surface patterns, including periodic patterns, are formed of raised structures having different dimensions, shapes, and spatial arrangements.

The raised structures of the present invention can be produced by numerous different techniques, such as photolithography, projection lithography, e-beam writing or lithography, depositing nanowire arrays, growing nanostructures on the surface of a substrate, soft lithography, replica molding, solution deposition, solution polymerization, electropolymerization, electrospinning, electroplating, vapor deposition, contact printing, etching, transfer patterning, microimprinting, self-assembly, and the like.

### BRIEF DESCRIPTION OF THE DRAWINGS

Various objects, features, and advantages of the present invention can be more fully appreciated with reference to the following detailed description of the invention when considered in connection with the following drawings, in which like reference numerals identify like elements. The following drawings are for the purpose of illustration only and are not intended to be limiting of the invention, the scope of which is set forth in the claims that follow.

FIG. 1A is a perspective view of an array of superhydrophobic posts.

FIG. 1B is a photograph of the top of an array of superhydrophobic posts with a droplet of an aqueous solution contacting the tops of the posts in the “Cassie” or “Cassie-Baxter” state, and FIG. 1C is a corresponding cross-sectional side view illustration which shows the droplet contacting the tops of the posts, including an exploded view of the vapor/liquid interface that defines a contact angle θ or θ*.

FIG. 1D is a side view illustration of an array of posts on a substrate (bottom) with an aqueous solution (top) in “Cassie” state and containing microorganisms that are only exposed to the tips of the structure.

FIG. 1E is a side view illustration of an array of posts on a substrate (bottom) with an aqueous solution (top) in partial or full transition to Wenzel (wetting) state and containing microorganisms, which adhere to posts wetted by the solution.

FIG. 1F depicts an illustration of a substrate having disordered raised structures, and

FIG. 1G depicts an illustration of a substrate having uniform or regular raised structures, demonstrating that the contaminated liquid (above the surfaces) is more efficiently excluded from the substrate subsurface by the uniform raised structures than by the disordered raised structures.

FIGS. 2A-2F are a series of illustrations and micrographs depicting a range of surfaces and their corresponding contact angles, including (a) flat hydrophilic substrates, (b) flat hydrophobic substrates, and (c)-(f) raised superhydrophobic structures on substrates including post arrays and brick raised surfaces (“intersecting walls”).

FIG. 3 depicts perspective, top- and side-view schematic diagrams of round raised post (3A), raised channel (“wall”) (3B) and raised closed-cell brick (“intersecting wall”) (3C) structures with widths (w), pitch (p) and interstructure spacings (s) indicated.

FIG. 4A is a schematic representing top views of different surface patterns formed by raised post structures, and illustrates the various cross-sectional shapes and areas of the posts as well as the variation in degree of ordering of the posts according to various embodiments.

FIG. 4B is a schematic representing top views of different raised channel structured surfaces according to various embodiments.

FIG. 4C are top view illustrations of substrates having raised closed-cell structures of various shapes with spaces between adjacent structures according to various embodiments.

FIG. 4D is a top view illustration of substrates having raised closed-cell structures with interconnecting walls, including brick compartments, square compartments, honeycomb compartments and web-patterned compartments according to various embodiments.

FIG. 5 A-D is a schematic representing cross-sectional views of reinforced branched I-shaped, (5A) T-shaped (5B), X-shaped (5C) and Y-shaped (5D) raised post structures according to various embodiments.

FIG. 5E depicts a scanning electron microscopy image of an array of exemplary array of branched T-shaped raised Si posts.

FIG. 5F depicts light microscopy image of an array of raised branched Y-shaped polymeric post structures with improved mechanical strength fabricated using molding.

FIGS. 6A-6F is a series of photographs depicting a droplet of an aqueous solution falling towards a surface (6A), impacting a superhydrophobic surface (6B), spreading (6C), and leaving the surface (6D-6F).

FIG. 7A is a side view illustration of a substrate having raised post structures having interstructure distances (s) less than about both the longest diameter d_{L} and shortest transverse diameter dₛ of the microorganism which preclude microorganisms from contacting the substrate.

FIG. 7B is a micrograph of B. subtilis on a substrate having raised post structures having interstructure distances less than the transverse diameter of the B. subtilis cells, demonstrating that the B. subtilis cells reside on the tips of the post structures and do not contact the substrate.

FIGS. 8A-8D are computer simulations of the mechanical properties of Si posts (width 1 micron, height 9 microns) having shapes of a straight cylinder (8A), branched Y-shaped (8B); branched T-shaped (8C); and having tapered, conical shape with basal width of 2.7 microns (8D), in which the insert provides a top plan view of the tested feature and the side bar graphs show the mechanical stress (von Mises, MPa) and displacement (μm).

FIGS. 9A-E are schematic cross-sectional views of five different arrays featuring raised post structures having widths which increase as they approach the basal surface from the distal ends.

FIG. 9F depicts scanning electron microscopy image of an array of raised conical Si post structures with improved mechanical strength fabricated using Bosch process.

FIG. 9G depicts scanning electron microscopy images of an array of raised conical Si post structures with improved mechanical strength fabricated using reshaping by electrodeposition of conducting polymers, at time (t)=0; at t=5 min; at t=10 min; at t=15 min; and t=20 min, demonstrating the formation of posts with increasingly wider bases

FIGS. 10A-10 F show optical and electron micrographs of exemplary raised closed-cell structures comprising honeycombs and brick walls according to one or more embodiments.

FIG. 11 is a schematic of a medical device with two types of patterned surfaces: in which (A) the top device is coated with a surface coating comprising raised structures, and (B) the bottom device itself has a surface comprising raised structures.

FIGS. 12A and 12B are illustrations of a method used to test the adhesion of bacteria to a substrate having raised superhydrophobic structures following contact with a contaminated liquid.

FIG. 13A depicts a flat hydrophobic (fluorinated) substrate (Si—F), a flat hydrophilic substrate (Si—C) on an agar plate; and FIG. 13B shows the corresponding agar plates after overnight culture.

FIG. 14A depicts an image of a substrate having both unpatterned (flat) and patterned raised post array surfaces; FIG. 14B depicts an image of the substrate face-down on an agar plate; and FIG. 14C depicts an image of the agar plate after overnight culture, showing an area corresponding to the patterned surface substantially free of microorganisms, while the area corresponding to the flat surface has significant microbial growth.

FIG. 15A-C depicts bacterial growth experiments after the exposure to the flow of contaminated liquid, as a function of the width of the raised posts.

FIG. 16A depicts an image of a substrate having both unpatterned (flat) and patterned region, bearing intersecting 1.3 micron-wide brick walls; FIG. 16B depicts an image of the substrate face-down on an agar plate; and FIG. 16C depicts an image of the agar plate after overnight culture, showing an area corresponding to the patterned surface free of microorganisms, while the area corresponding to the flat surface has significant microbial growth.

FIG. 17A depicts images of E. coli bacteria growing on flat (unpatterned) surfaces; and FIG. 17B depicts images of surfaces bearing raised post structures with interstructure spacings of less than the smallest dimension of the E. coli, showing no E. coli present on the tops of the posts; in which the top images are electron micrographs and bottom images are optical micrographs.

### DETAILED DESCRIPTION OF THE INVENTION

All publications, patent applications, patents, and other references mentioned herein, are incorporated by reference in their entirety. Unless otherwise defined, all technical and scientific terms used herein have the same meaning as commonly understood by one of ordinary skill in the art to which this invention belongs. In case of conflict, the present specification, including definitions, will control. In addition, the materials, methods, and examples are illustrative only and not intended to be limiting. Although methods and materials similar or equivalent to those described herein can be used in the practice or testing of the present invention, suitable methods and materials are described below.

Other features and advantages of the invention will be apparent from the following detailed description, and from the claims.

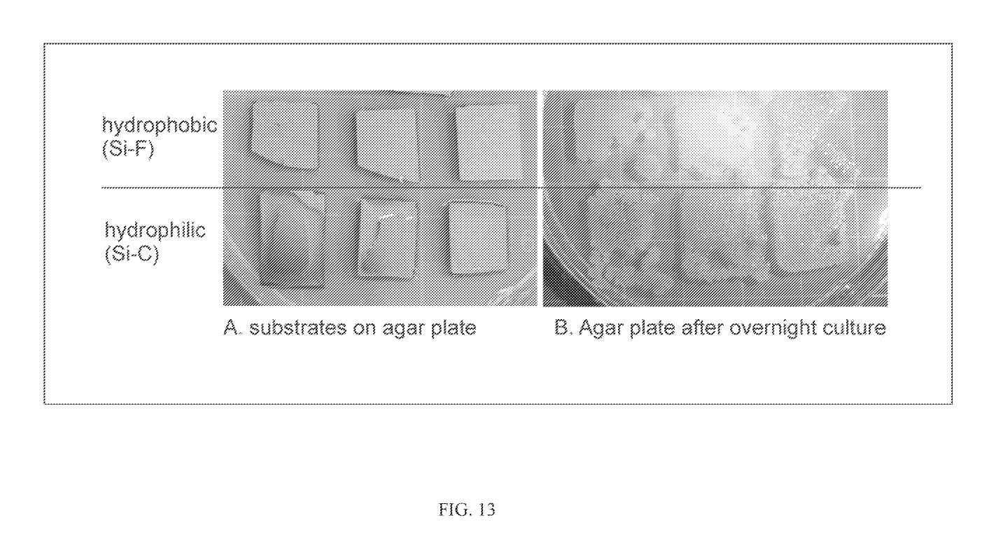

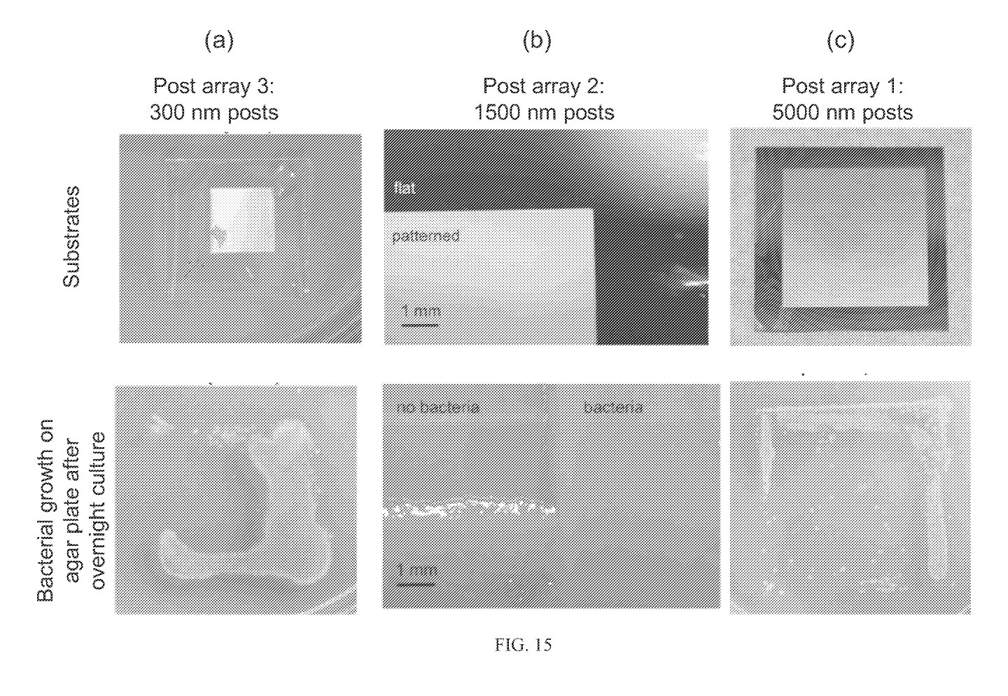

Raised structures, and methods of using such structures to prevent, inhibit, or reduce the attachment of microorganisms onto substrates, are described. Such raised structures prevent, inhibit, or reduce the attachment of microorganisms on substrates when contacted with contaminated liquids containing a microorganism. The contact can be static, due to simple or ongoing exposure to a contaminated liquid, or dynamic, such as contact due to splashing or pouring of the microorganism-containing liquid. In some embodiments, the adhesion is fully inhibited or reduced following temporary contact of the contaminated fluid. In some embodiments, the contact lasts a few milliseconds to a few minutes. The surfaces thus exposed either remain sterile or result in loosely attached microorganisms that can be readily removed by physical or chemical treatment. Sterile surfaces either are completely sterile (completely free of microorganisms), effectively sterile (containing sufficiently few loosely attached or poorly organized organisms that no microorganisms are transferred from that surface to another environment), or exhibit limited or reduced contamination (microorganism attachment is less than a comparable surface lacking raised structures). Sterility or microorganism contamination is measured by any of numerous methods known to those in the art, such as by measuring the number of colony forming units (CFU) present per volume of liquid or mass of solid using the Miles and Misra method described in Miles, A. A; Misra, S. S. J. Hyg. (London), 38, 732 (1938), hereby incorporated by reference in its entirety. Sterility or microorganism contamination can also be measured by image analysis of the extent of microorganism growth on a surface. For example, image analysis was conducted on images of the agar plates shown in FIGS. 13B, 14C, 15A, B, and C (lower images), and 16C. The flat control surfaces shown in these figures transfer a printed (contaminated) area that, after growth, constituted between 75% to 100% of the original area, while superhydrophobic surfaces having raised posts and walls with diameters and/or widths of less than about 2 microns transferred areas of 0% of the original area (i.e. no microorganism growth). See, e.g., FIGS. 15A-B, 16C. Superhydrophobic surfaces having raised posts with diameters of 5 microns transferred an area of approximately 4% of the original area (i.e. some microorganism growth). See, e.g., FIG. 15C. Other methods of measuring sterility or microorganism contamination are discussed in Christensen, G. D., et al. J. Clin. Microbiol. 22, 996-1006 (1985); Costerton, J. W., et al. Ann. Rev. Microbiol. 41, 435-464 (1987); Gristina, A. G. Science 237, 1588-1595 (1987); Everaert, E. P. J. M., van der Mei, H. C. & Busscher, H. J. Colloids and Surfaces B: Biointerfaces 10, 179-190 (1998); Jacques, M., Marrie, T. J. & Costerton, J. W. Microbial Ecology 13, 173-191 (1987); Hall, L. B. & Hartnett, B. A. Public Health Records 79, 1021-1024 (1964); Druskin, M. S. & Siegel, P. D. J. Am. Med. Assoc. 185, 966-968 (1963); Bentley, D. W. & Lepper, M. H. J. Am. Med. Assoc. 206, 1749-1752 (1968); Corso, J. A., Agostinelli, R. & Brandriss, M. W. J. Am. Med. Assoc. 210, 2075-2077 (1969); Irwin, G. R., Hart, R. J. & Martin, C. M. Pathogenesis and Prevention of Intravenous Catheter Infections. Yale Journal of Biology and Medicine 46, 85-93 (1973); Michel, L., McMichan, J. C. & Bachy, J.-L. Am. J. Surgery 137, 745-748 (1979); Shinozaki, T., Deane, R. S., Mazuzan, J. E., Hamel, A. J. & Hazelton, D. J. Am. Med. Assoc. 249, 223-225 (1983); Crnich, C. J. & Maki, D. Clinical Infectious Diseases 34, 1232-1242 (2002); and Genzer, J. & Efimenko, K. Biofouling 22, 339-360 (2006); hereby incorporated by reference in their entireties.

In some embodiments, the raised structures are posts. In further embodiments, the raised structures are channels. In still further embodiments, the raised structures are closed-cell structures. In still further embodiments, the raised structures are a combination of the above.

In one aspect, the treated surface comprises raised superhydrophobic structures which energetically exclude microorganisms by preventing wetting of the surface by a contaminated liquid under dynamic conditions, such as pouring, splashing or sprinkling of the liquid on the surface.

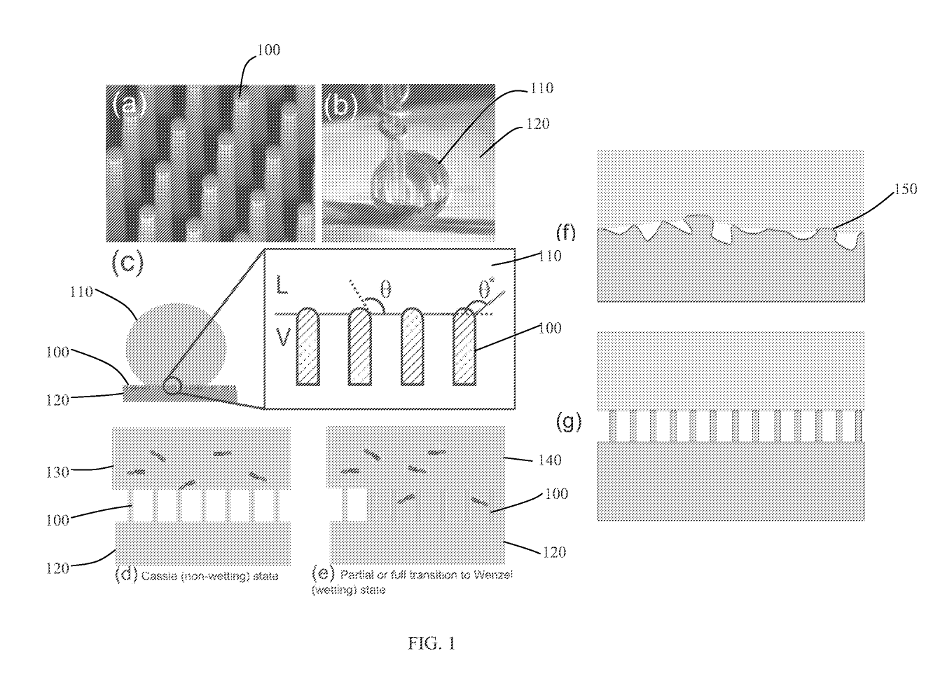

As used herein, “superhydrophobic” means a surface that is highly hydrophobic and non-wetting, with the liquid/surface interface having a contact angle θ of at least about 140°, and the liquid in the so-called “Cassie” state such that the liquid is only in contact with the tips of the raised surface features and is resting on a cushion of air. The contact angle (θ), as seen in FIG. 1C, is the angle at which the liquid-vapor interface meets the solid-liquid interface. The tendency of a drop to spread out over a flat, solid surface increases as the contact angle decreases. Thus, the contact angle provides an inverse measure of wetability

FIG. 1A shows exemplary superhydrophobic surface having an array of posts 100. The posts are hydrophobic, e.g., they can be made of a material that is hydrophobic or coated or chemically treated to provide a hydrophobic surface. Liquids, e.g., water, bead up and do not wet the surface of the superhydrophobic surface. FIG. 1B shows non-wetting water droplet 110 on a superhydrophobic surface 120 made up, for example, of an array of posts 100, such as are shown in FIG. 1A. FIG. 1C shows a cross-sectional view of the water droplet as it rests on the microstructured superhydrophobic surface. FIG. 1C also provides a magnified view of the relative positions of the liquid phase (L), vapor (V) on a substrate. In this figures, θ is the contact angle for a Cassie state liquid, and θ* is the apparent contact angle which corresponds to the stable equilibrium state. Superhydrophobic surfaces are known in the art, and are known to be influenced by factors such as, but not limited to, the surface composition, the widths, heights, and interstructure spacings of the raised surfaces. One of skill in the art will appreciate how these factors influence the contact angle exhibited by a surface.

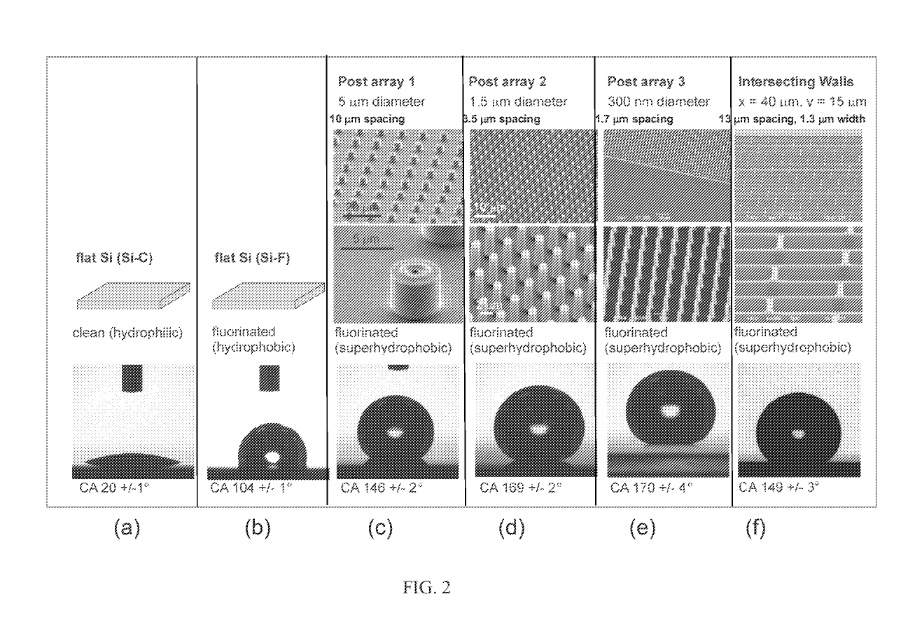

FIG. 2A-2F illustrate how the properties of various surfaces affect the contact angle of the liquid/surface interface. FIG. 2 depicts flat (a) hydrophilic and (b) hydrophobic surfaces lacking raised structures, exhibiting contact angles of less than 140°, demonstrating that coating a flat surface with a hydrophobic material is not by itself enough to produce a superhydrophobic surface. Images (c)-(f) depict fluorinated surfaces bearing raised structures of (c)-(e) post arrays and (f) closed-cell brick (or “intersecting wall”) structures of varying widths (or “diameters”) and pitches exhibiting contact angles of greater than 140°, showing that raised surfaces of described herein produce superhydrophobic surfaces. In each of the figures, the upper image is a low magnification image of the surface, the center image is a high magnification image of the surface and the lower image is of a water droplet on the surface indicating contact angle. FIG. 2C shows a regular post array of 5 μm wide posts with an interpost spacing of 10 μm. A water droplet on the surface has a contact angle of 146°. Decreasing both the post size and interpost spacing increases the hydrophobicity of the surface and the contact angle of a water droplet (169°), as shown in FIG. 2D. Still smaller diameter posts (300 nm) and interpost spacings (1.7 μm), provide about the same contact angle (FIG. 2E). Interestingly the projected surface areas (phi ratio) of the posts in FIGS. 2D and 2E are similar. Lastly, FIG. 2F demonstrates that raised surface features other than posts can also form superhydrophobic surfaces and large contact angles (e.g., 149°).

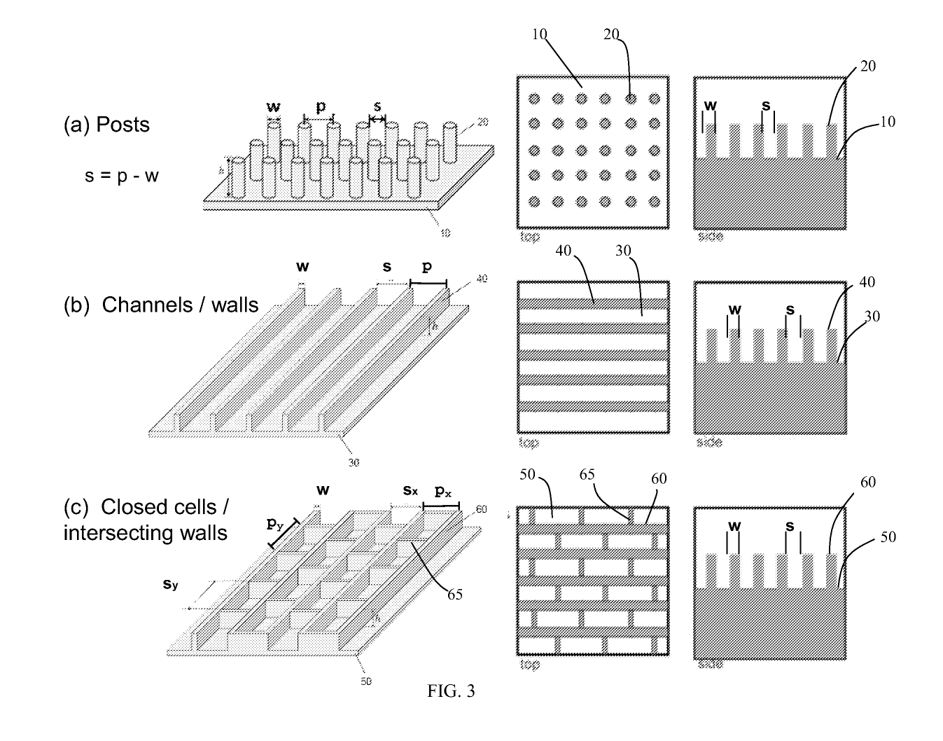

FIG. 3A shows a post array having posts 20 on subsurface 10 in perspective, plan and cross-sectional views. FIG. 3B shows a channel array having walls 40 on subsurface 30 in perspective, plan and cross-sectional views. Lastly, FIG. 3C shows a closed-cell array having long walls 60 and transverse short walls 65 on subsurface 50 in perspective, plan and cross-sectional views. As used herein, “width” (w) refers to the shortest transverse distance of the distal ends of a raised surface. For example, FIG. 3 shows that the width of the distal end of a raised circular post surface is its diameter at its distal end (3A), and the width of the distal end of a raised surface defining channels or closed-cell structures is the width of the wall defining the channel or closed-cell structure at its distal end (3B and 3C, respectively).

As used herein, “pitch” (p), or periodicity, refers to the distance between the centers of adjacent raised structures. For example, FIG. 3 shows that the pitch between posts is the distance between the centers of adjacent posts (3A), the pitch between raised structures defining channels is the average distance between the centers of adjacent lateral walls (3B), and the pitch between raised structures defining closed-cell structures is the average distance (per compartment) between the centers of the wall or opposite walls delimiting the closed-cell structure (e.g., for some symmetric compartments such as those exhibiting square, hexagonal, octagonal, etc. geometry, the interstructure spacings would be equal to the distance between the centers of oppositely facing lateral walls; for non-symmetric compartments: pₓ and p_{y}).

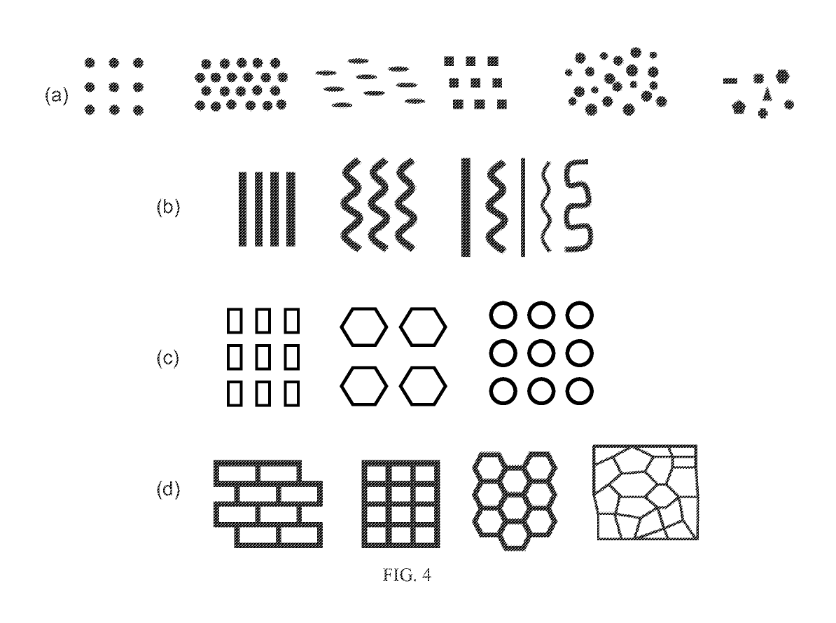

As used herein, “interstructure spacing” (s) refers to the shortest lateral dimension of the available space/gap between adjacent raised structures. FIG. 3A-B show that the interstructure spacing is equal to the pitch minus width of the structures. For structures with varying interstructure spacings, such as non-uniformly spaced posts, non-symmetric compartments, and non-symmetric channels as seen in some aspects of FIGS. 4A-B and 4D, interstructure spacing is better defined as the average shortest available space/gap between adjacent raised structures per compartment.

In another aspect, the surface comprises raised structures having raised structures having both a width and interstructure spacing of less than about the length and/or transverse diameter of the microorganism contained in the contaminated liquid, which physically exclude microorganisms from the substrate subsurface. In some embodiments, the microorganism contacts the tops of the structures, such as at a reticulated surface made up of the tops of the structures, and does not contact the base or sub-substrate.

In some embodiments, the microorganism is an aspected microorganism, e.g., a rod-shaped microorganism, having a length and a transverse diameter. In other aspects, the microorganism is a non-aspected microorganism, e.g., a spherical microorganism, having a diameter.

In some embodiments, the microorganism is a biofilm-forming microorganism, and biofilm formation is inhibited, delayed, or attenuated, according to the methods described herein.

In some embodiments, a substrate used to reduce or inhibit the attachment of microorganisms includes raised structures that can vary in dimensions, shape, and spatial arrangement. In some embodiments, the heights and widths of the raised structures on the substrate are uniform. In further embodiments, the heights and widths of the raised structures vary across the substrate. In some embodiments, the heights of the raised structures change gradually across the substrate, e.g., creating a gradient of heights. In further embodiments, the heights of the raised structures vary randomly across the substrate. Similarly, in some embodiments the widths of the raised structures on the substrate are uniform. In further embodiments, the widths of the raised structures vary across the substrate. In some embodiments, the widths of the raised structures change gradually across the substrate, e.g., creating a gradient of widths. In further embodiments, the widths of the raised structures vary randomly across the substrate. In some embodiments, the shapes of the raised structures on the substrate are uniform. In further embodiments, the shapes of the raised structures vary across the substrate. In some embodiments, the shapes of the raised structures change gradually across the substrate, e.g., creating a gradient of shapes. In further embodiments, the shapes of the raised structures vary randomly across the substrate. In some embodiments, the interstructure spacings of the raised structures on the substrate are uniform or regular. In further embodiments, the interstructure spacings of the raised structures vary across the substrate. In some embodiments, the interstructure spacings of the raised structures change gradually across the substrate, e.g., creating a gradient of interstructure spacings. In further embodiments, the interstructure spacings of the raised structures vary randomly across the substrate. In some embodiments, the raised structures are distributed in an ordered fashion, e.g., symmetrically arranged. In further embodiments, the raised structures are randomly positioned.

In some embodiments, the raised structures are either isolated or interconnected. Thus, different surface patterns, including periodic patterns, are formed of raised structures having different dimensions, shapes, and spatial arrangements, as exemplified in FIGS. 4A-4D. The contaminated liquid (above the surfaces) is more efficiently excluded from the substrate subsurface by the uniform raised structures than by the disordered raised structures, as shown in FIG. 1F-G; therefore, uniform raised structures are preferred.

In some embodiments, the width of the raised structures are selected to prevent or discourage microorganism attachment to the surface. In some embodiments, the width of the raised structures are less than or about 5 μm. In some embodiments, the width of the raised structures are less than or about 2 μm. In some embodiments, the width of the raised structures is in the range of about 5 μm to about 100 nm, or about 2 μm to about 300 nm. In some embodiments, the width of the raised structures are less than about the smallest axis of a microorganism. In further embodiments, the width of the raised structures are less than about the length of a microorganism or less than about the diameter of a microorganism.

Viruses are very small and range from about 20 to 250 nm in dimension. Fungal spores are in the range of 1-100 microns (and most between 2-20 microns), and bacterial spores are in the range of 0.5 to 2 microns. Feature dimensions can be determined accordingly For example, for bacteria and fungi, the upper limit to post dimensions can be in the range of about 3-5 times the size of the organism, which in many cases allows for prevention or discouragement of bacterial and fungal attachment to the surface using post dimensions of about 3-5 microns. Experimental results have demonstrated that 5 micron posts are at a size range that results in little to no microorganism contamination and/or prevents biofilm formation on the treated surface.

In certain embodiments, the raised structures are generally vertically oriented to the substrate (e.g., perpendicular). In further embodiments, the raised structures are oriented oblique to the substrate.

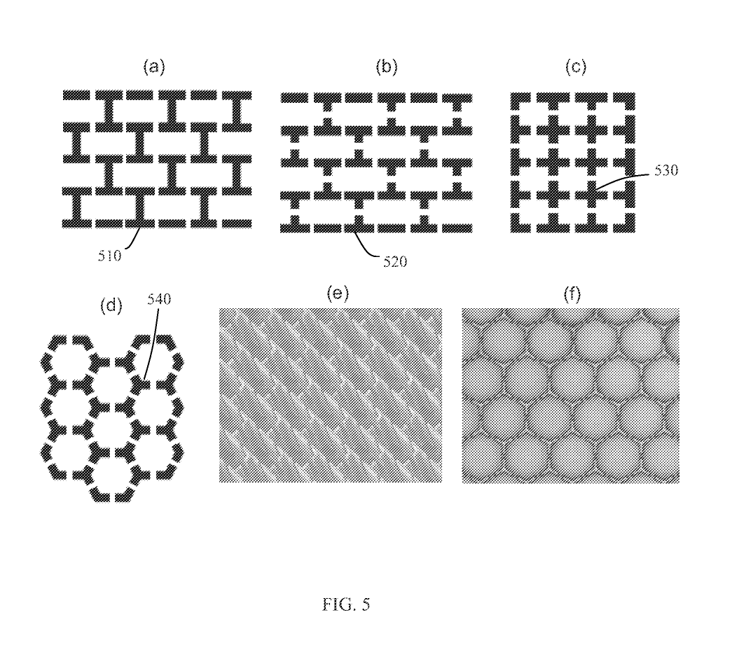

In some embodiments, the raised post structures comprise mechanically reinforced posts, having branched cross-sections for mechanical stability. For example, FIGS. 5A-5D shows that such posts can have branched T-shaped, Y-shaped, or X-shaped cross-sections, or branched I-beam shapes, known to be used in construction due to their maximum mechanical stability. In further embodiments, posts can be S-shaped in cross section.

In some embodiments, the raised structures comprise mechanically reinforced structures, having basal widths greater than their distal widths.

In some embodiments, the raised structures are prepared as a coating on a device, such as a medical device, to prevent, inhibit, or reduce the attachment of microorganisms on to the device. In further embodiments, the surface itself is structured so as to define the raised structures described herein.

The raised structures of the present invention can be produced by numerous different techniques, such as photolithography, projection lithography, e-beam writing or lithography, depositing nanowire arrays, growing nanostructures on the surface of a substrate, soft lithography, replica molding, solution deposition, solution polymerization, electropolymerization, electrospinning, electroplating, vapor deposition, contact printing, etching, transfer patterning, microimprinting, self-assembly, and the like.

Energetic Exclusion of Microorganisms

The invention is based in part on the discovery that superhydrophobic raised structures having defined feature sizes can be used to fully inhibit or reduce the adhesion of microorganisms on a substrate upon dynamic impact (such as by splashing, pouring, or sprinkling) of a contaminated liquid containing microorganisms on the surface.

In one aspect, the raised structures comprise raised superhydrophobic structures that provide a surface that energetically excludes microorganisms by preventing wetting of the surface by a contaminated liquid under dynamic conditions (such as pouring, splashing, or sprinkling of the liquid on the surface). In some embodiment, the structures have a width of less than about 5 μm to prevent bacterial attachment and less than about 15 μm to prevent fungal attachment.

In some embodiments, the width of the raised structures are selected to prevent or discourage microorganism attachment to the surface. In some embodiments, the width of the raised structures are less than or about 5 μm for bacteria or viruses. For fungal organisms, the feature width can be less than or about 10 μm. In some embodiments, the width of the raised structures are less than or about 2 μm. In some embodiments, the width of the raised structures is in the range of about 5 μm to about 100 nm, or about 2 μm to about 300 nm. In some embodiments, the width of the raised structures are less than about the smallest axis of a microorganism. In further embodiments, the width of the raised structures are less than about the length of a microorganism or less than about the diameter of a microorganism.

When the feature diameters are at or less than the dimensions of the microorganism, the microorganisms have difficulty attaching to the tops of the raised surface features. When the surface is in a Cassie state so the contact angle of liquids is high and the contact area is low, the ability of the microorganisms to attach and proliferate on the surface is further hindered. In one or more embodiments, the feature dimensions prevent biofilm formation. In some embodiments, raised structures having widths of less than about 2 microns result in effectively sterile surfaces under dynamic conditions (pouring, sprinkling, or splashing by a contaminated liquid). In further embodiments, raised structures having widths of between about 2 and about 20 microns result in surfaces exhibiting limited or reduced contamination under dynamic conditions (pouring, sprinkling, or splashing by a contaminated liquid).

To minimize the contact area between a water droplet and a patterned hydrophobic surface, the possibility that the droplet remains in the so-called “Cassie-Baxter” state, i.e., non-wetted state, without transitioning to the so-called Wenzel state, i.e., wetted state must be maximized. Note that a droplet in the “Cassie-Baxter” state only wets the tops of raised structures, thereby minimizing the contact area. In contrast, a droplet in the Wenzel state wets the entire surface, e.g., the top surfaces of the raised features as well as the subsurface to which the raised features are attached. For a discussion of these two states, see, e.g., Cassie et al. Trans. Faraday Soc., 1944, 40, 546-550 and Wenzel, J. Phys. Colloid Chem., 1949, 53, 1466-1467, which is hereby incorporated by reference in its entirety. To maximize the possibility that a droplet stays in the “Cassie-Baxter” state, the size of raised structures can be decreased to appropriate dimensions on a hydrophobic surface, thereby further increasing the hydrophobicity of the surface. Indeed, this approach allows preparation of a superhydrophobic surface, i.e., a surface on which a water droplet has a contact angle equal to or greater than 140°. Note that the larger the contact angle, the smaller the contact area. The substrate can be made of a hydrophobic material that reinforces the superhydrophobic effect of the raised surfaces.

Superhydrophobic surfaces, such as an array of hydrophobic posts, are non-wetting for contaminated liquids such that droplets in the so-called “Cassie” state are only in contact with the very top features of the surface structures (see Danese, Chemistry and Biology 9, 873-880 (2002); Crnich et al., Clinical Infectious Diseases 34, 1232-1242 (2002); Crnich et al., Clinical Infectious Diseases 34, 1362-1368 (2002); Genzer et al., Biofouling 22, 339-360 (2006); Callies et al., Soft Matter 1, 55-61 (2005); Barthlott et al., Planta 202, 1 (1997), hereby incorporated by reference in their entireties). This is shown in FIGS. 1B and 1C which show the droplet contacting the tops of the posts and defining a contact angle of the surface.

FIG. 1D shows a superhydrophobic surface having raised posts 100 on a subsurface 120 and illustrates the confining effect of a superhydrophobic surface on microorganism attachment. Microbial organisms in solution 130 only have limited contact with the surface (FIG. 1D). However, extended exposure times can cause partial or complete wetting 140 of the surface (FIG. 1E). Therefore, the lifetime of the “Cassie” state can be limited, and the prospects of the non-wetting contact of contaminating liquids (i.e., liquids containing microorganisms) can also be limited.

Since there exists an induction time for microorganism attachment (i.e., the time required for a microorganism to attach to the raised surface or substrate), conditions for which water droplets bounce off a surface before microorganism attachment occurs can be created. Contaminated liquid droplets bounce off a patterned superhydrophobic surface and their contact time with the surface is shorter than the time required for microorganism attachment. In contrast, contaminated liquid droplets typically do not bounce off unpatterned hydrophobic surfaces, or patterned or unpatterned hydrophilic surfaces. As a result, such droplets can remain in contact with unpatterned hydrophobic or any hydrophilic surfaces and provide sufficient opportunity for microorganisms to attach to these surfaces.

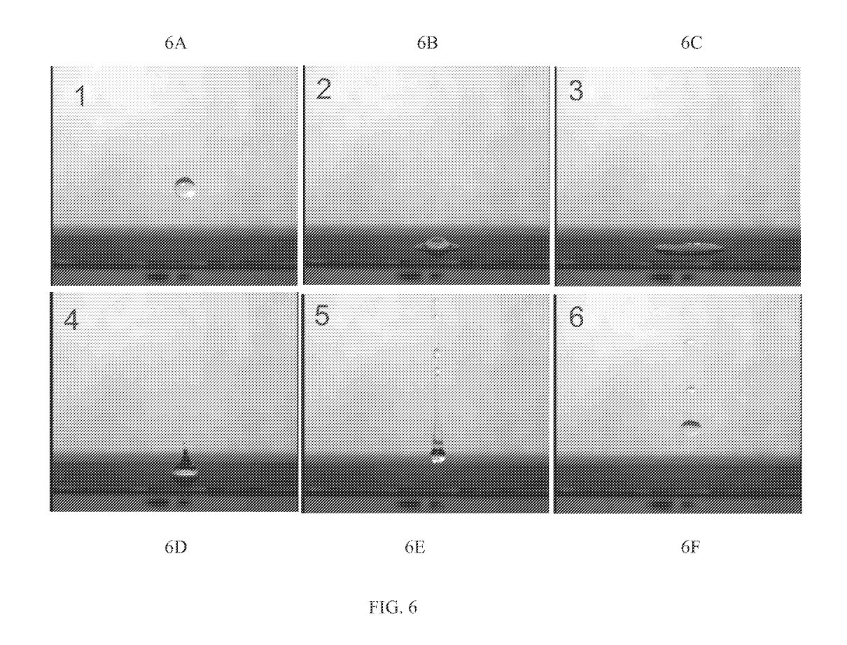

An important consequence of superhydrophobicity is that impacting droplets will spread, but then retract and rapidly de-wet from the surface altogether (see Feng et al., Advanced Materials 18, 3063-3078 (2006); Quere, Ann. Rev. Mater. Res. 38, 71-99 (2008); Richard et al., Europhys. Lett. 50, 769-775 (2000); Richard et al., Nature 417, 811 (2002); Bartolo et al., Europhys. Lett. 74, 299-305 (2006), hereby incorporated by reference in their entireties). Such impacting droplets only remain in contact with the surface for a limited time, which is mostly a function of the droplet size and not of the droplet impact velocity (see Quere, Ann. Rev. Mater. Res. 38, 71-99 (2008), hereby incorporated by reference in its entirety), and which is on the order of 10¹ to 10² milliseconds, for droplets of size 1-3 mm. FIG. 6 shows a series of photographs depicting (6A) a droplet of an aqueous solution (6B) impacting a superhydrophobic surface, (6C) spreading, and (6D)-(6F) then completely de-wetting from (i.e., leaving) the surface.

The property of fast droplet de-wetting and ejection from a surface, in combination with superhydrophobic raised structures of defined feature size that interferes with the ability of bacteria, viruses or fungi, contained within such a droplet, to physically attach to the surface, provides a surface that is resistant to cell attachment and biofilm formation. Therefore, after droplet de-wetting and ejection from the surface by a contaminated liquid, there are either no or very few loosely attached or poorly organized microorganisms left behind. As a result, the complete or substantial absence of microbial organisms means that the surface remains either completely sterile (completely free of microorganisms) or effectively sterile (containing sufficiently few loosely attached or poorly organized organisms that no microorganisms are able to be transferred from that surface to another environment). The inability of bacteria to attach to the surface is a combination of the factors of limited time for surface contact of the droplet, and the very limited surface area for bacterial or fungal attachment.

In certain embodiments, the raised superhydrophobic structures are prepared from a hydrophobic material, and/or include a hydrophobic coating. In some embodiments, the raised superhydrophobic structures are fluorinated. In particular embodiments, the raised superhydrophobic structures (or the array of raised superhydrophobic structures) have a contact angle of greater than about 140°, such as between about 150° and about 180°.

Physical Exclusion of Microorganisms

Further, it has been discovered that interstructure spacing, dimension and geometry of raised structures can be used to inhibit, reduce, or attenuate microorganism attachment.

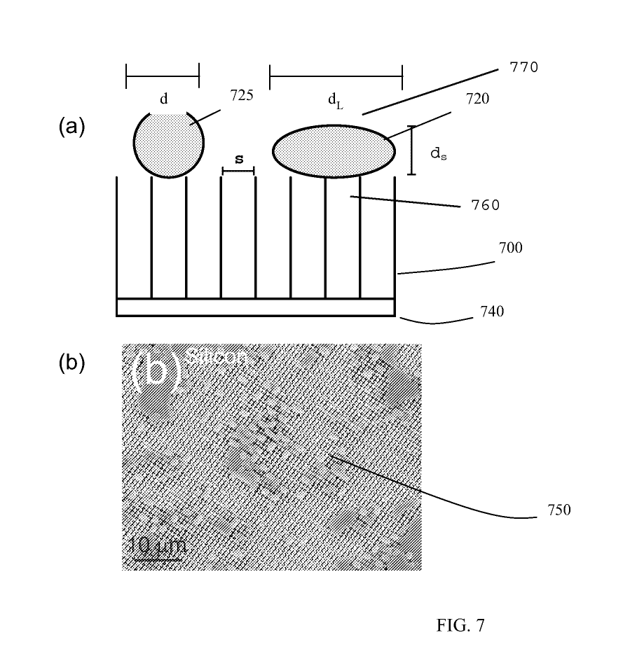

In another aspect, the raised structures can have interstructure spacings of less than about the length and/or transverse diameter of the microorganism contained in the contaminated liquid, which physically exclude microorganisms from the substrate subsurface. In these embodiments, the interstructure spacing is too small to permit the microorganisms to enter the interstructure space and attach to the base surface, and they are instead constrained to the upper surface of the raised structures. For example, FIG. 7A shows a side view of a substrate 740 having raised post structures 700 with interstructure distances s less than about the transverse diameter d of a microorganism, 725, so that the microorganism is precluded from contacting the substrate. An aspected microorganism 720 is also shown, having a shortest transverse diameter dₛ and a longest diameter d_{L}. As microorganisms 720 and 725 are constrained to the upper surface of the raised structures, these microorganisms are more susceptible to biological or chemical attack, as they can be accessed from both the top 770 and the available space below 760. FIG. 7B is a micrograph of B. subtilis microorganisms on such a substrate, where the cells 750 reside on the tips of the post structures and do not contact the substrate. Thus, even when there is a possibility of surface wetting, so that the liquid contacts the surface for a time sufficient to permit attachment, little or only weak attachment occurs. In some embodiments, the raised structures are not superhydrophobic. In further embodiments, the raised structures are hydrophilic.

In the instance of physical exclusion of the microorganism, for example when the bacteria is attached only to the tips of the raised features, it can be removed by mechanical or chemical means, much more easily than for a flat surface. While the ease of the mechanical removal is due to the limited surface contact and reduced adhesion, also chemical or biological removal is simplified due to the fact that having a porous volume underneath the microorganism (e.g., bacterial biofilm) provides a means to attack the microorganism not only from the top (e.g., as in biofilms formed on flat surfaces), but also from the bottom (e.g., by antibiotics or other chemical means, whether liquid or gaseous) will have the access to the bottom part of the microorganism as well increasing the surface area of attack.

In some embodiments, the interstructure spacings of the raised structures are less than about the smallest axis of a microorganism. In further embodiments, the interstructure spacings of the raised structures are less than about the length and greater than about the transverse diameter of a microorganism. In further embodiments, as the interstructure spacings of the raised structures decrease and are less than about the shortest dimension of a microorganism, the microorganism contacts the tips of the structures and does not contact the substrate.

As noted above, the diameters of the raised features can also be selected to discourage microorganism adhesion. Typically, a rod-shaped microorganism has a length of about 0.1 μm to about 10 μm or longer and a transverse diameter of about 0.1 μm to about 5 μm or wider. A spherical microorganism can have a diameter of about 0.1 μm to about 1 μm. Accordingly, raised structures disposed on substrates can have widths based on the lengths and/or diameters of a particular microorganism. For example, Pseudomonas aeruginosa (strain PA14), the cause of most hospital-acquired diseases, has a lateral length of about 1 μm to about 2 μm and a transverse diameter of about 0.5 μm to about 1 μm. For this microorganism, a substrate having raised structures with widths of less than about 2 μm inhibit or reduce the attachment of this microorganism, while a substrate having raised structures with interstructure spacings of less than about 0.5 μm would control the microorganism such that the microorganism would be confined to the tops of the raised structures.

In certain embodiments, the microorganism is a biofilm-forming microorganism, and the arrangement of the microorganism is controlled such that the formation of a biofilm is inhibited, delayed, or attenuated. For example, where a biofilm is formed by a microorganism on a substrate described herein, such biofilm is attenuated and can be easily removed from the substrate, such as by rinsing or washing, due to the fact that it is suspended at the tips of the structure thus having a limited contact with the surfaces.

In certain embodiments, the surface is a superhydrophobic surface having raised features with diameters of less than about 10 μm (for fungus) or less than about 5 μm (for bacteria or viruses) or less than or about 2 μm, so that the surface contact area is low and liquid have low residence times of the surface. Microorganism adhesion is further reduced or prevented by providing an interstructure spacing of less than about 2 μm inhibit or with interstructure spacings of less than about 0.5 μm to confine the microorganism to the tops of the raised structures. The particular features of the antibiofilm surface is dependent on the microbial system. Surface features having a distal width of 5 μm or less will work for most bacterial systems (and therefore fungal, as fungi are larger than bacteria). However, depending on the nature of the exposure, additional feature sizes may be preferred.

In certain embodiments, the surfaces reduce the attachment of bacteria/fungi during energetic exclusion (splashing) when the width is smaller than about 3-5 times the size of the bacterial/fungal cell (as in Example 3, where 5 micron posts do not fully prevent, but reduce the attachment).

In other embodiments, the surfaces remain fully sterile during energetic exclusion (splashing) when the width of the features are less than the size of the bacteria (about 1.5 microns in Example 3.

In still other embodiments, the surfaces physically exclude bacteria at long exposures when the gap is smaller than the smallest dimension of the bacterium, fungus, or virus and the microorganism (e.g., a bacterial film) then forms at the tips with limited contact (resulting in easier physical or mechanical removal) and easy accessibility from the bottom (resulting in greater susceptibility to chemical or biological treatment from the porous volume underneath the microorganism for diffusing chemical or biological species).

In other embodiments, the surfaces both energetically and physically exclude bacteria at either splashing or at long exposures when both the width and the gap are smaller than the smallest dimension of the bacterium/fungus.

Upon culturing the surfaces (or articles containing a coating layer with the raised features) after exposure to a contaminated fluid, under the conditions described in Example 3, the surface did show evidence of biofilm growth.

Improved Mechanical Strength of Raised Structures

Traditional structured surfaces mostly made up of an array of pillars can be easily damaged by impact and scratch, and when so damaged lose any properties imbued by the raised structures. The raised structures of the present invention provide structured surfaces with the desired anti-wetting and/or cell exclusion properties, but with improved mechanical strength and impact resistance.

The raised structures according to one or more embodiments display high mechanical stability and scratch resistance. Posts are most susceptible to damage, as they have relatively small dimensions in all directions. Channels and closed cell structures are somewhat stronger, as they have extended dimensions in at least one dimension, e.g., length, and even cross feature reinforcement in the case of closed cell structures.

In some embodiments, the raised structures, including raised post structures, are further strengthened by providing basal widths greater than distal widths. In some embodiments, reinforced post structures display increased mechanical stability and scratch resistance due to branched I-, Y-, T- or X-shaped columns, or posts with S-shaped cross sections. These geometries have improved mechanical properties compared with cylindrical or polygonal columns. In some embodiments, raised post structures have these improved mechanical strengths due to branched cross-sections (e.g., branched T-shaped, Y-shaped, or X-shaped cross-sections, or branched I-beam shapes) or non-linear cross sections (e.g., S-shaped cross sections). The branched cross-sectioned featured can be even further strengthened by grouping or arranging the branched posts into arrangements that mimic closed cell structures. For example, in FIG. 5A, branched I-beam shaped columns 510 are arranged in groups to approximate the geometry of a ‘brick’ closed cell structure. Similarly, in FIG. 5B, branched T-shaped columns 520 are arranged in groups to approximate the geometry of a ‘brick’ closed cell structure. Branched X-shaped columns 530 can be arranged to form closed-cell structures having square cells (FIG. 5C), while branched Y-shaped columns 540 can be arranged to form a closed-cell honeycomb structure.

In still further embodiments, channeled structures of the present invention have these improved properties due to reinforced sinusoidal, wavy, or zigzag walls (FIG. 4B). In yet further embodiments, closed-cell structures have these improved properties due to interconnected supported walls.

In some embodiments, the reinforced raised structures of the present invention having basal widths greater than distal widths demonstrate at least a two-fold improvement in maximum shear stress before mechanical failure (e.g., fracture) over analogous non-reinforced structures (structures having basal widths not greater than the same distal widths of the reinforced structures). In further embodiments, the improvement is at least three-fold. In still further embodiments, the improvement is at least four-fold

In some embodiments, the reinforced raised structures of the present invention having branched T-, I-, X-, and Y-shaped raised post structures or S-shaped in cross section demonstrate at least a two-fold improvement in maximum shear stress before mechanical failure (e.g., fracture) over analogous non-reinforced structures (structures lacking branching). In further embodiments, the improvement is at least three-fold. In still further embodiments, the improvement is at least four-fold.

In some embodiments, the reinforced raised structures of the present invention have a strength (maximum shear stress before mechanical failure, e.g., fracture) of greater than 10 MPa. In further embodiments, the strength of the reinforced raised structures is greater than 50 MPa. In still further embodiments, the strength of the reinforced raised structures is greater than 100 MPa. In still further embodiments, the strength of the reinforced raised structures is greater than 200 MPa. In still further embodiments, the strength of the reinforced raised structures is greater than 300 MPa. In other embodiments, the strength of the reinforced raised structures is in the range of about 100-500 MPa, or 200-400 MPa, or 300-400 MPa,

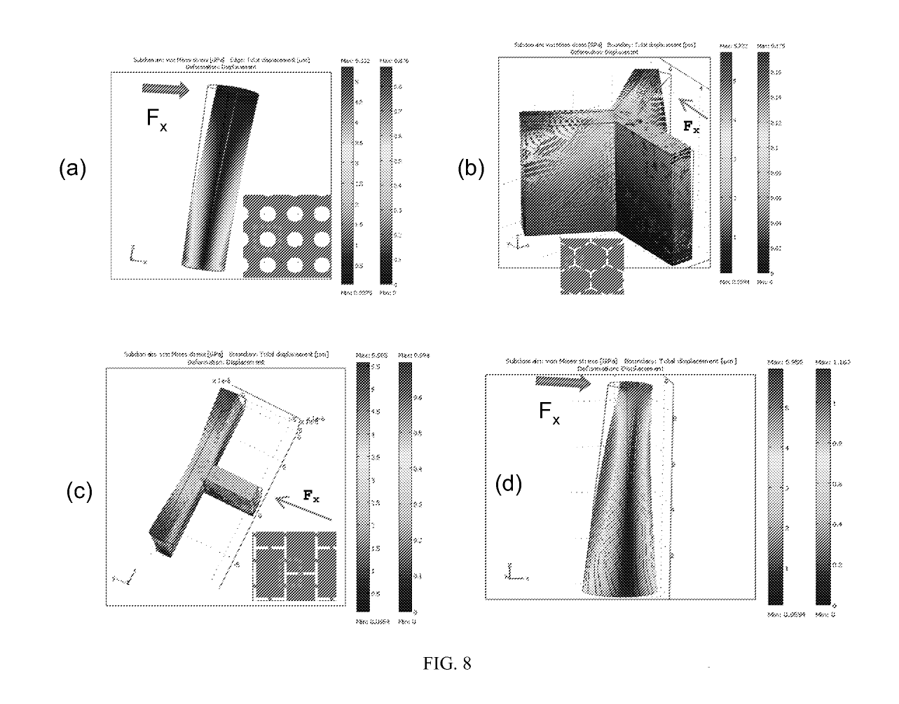

FIG. 8 shows computer simulation results demonstrating that the forces required to break exemplary branched T- and Y-shaped raised posts are at least 3-4 times higher than those that would break raised cylindrical posts of the same dimensions. FIG. 8A presents the stress field for a cylindrical Si column with the width of 1 μm, height 9 μm. A plan view of an array of such columns is shown in the inset of FIG. 8A. The maximum shear stress applied at the top of such structure (indicated by arrow Fₓ) is about 100 MPa before mechanical failure (e.g., fracture). FIG. 8B presents the stress field for a branched Y-shaped Si column with the same width of 1 μm, height 9 μm. The columns are arranged in a honeycomb geometry as shown in the plan view of an array of such columns in the inset of FIG. 8B. The maximum shear stress at the top of such structure is about 350 MPa before mechanical failure (e.g., fracture), more than a 3-fold increase over a simple column. FIG. 8C presents the stress field for a branched T-shaped Si column with the similar width of 1 μm, height 9 μm. The maximum shear stress at the top of such structure is about 300 MPa. The columns are arranged in a brick geometry as shown in the plan view of an array of such columns in the inset of FIG. 8C. This stress field model shows an at least about threefold improvement in maximum shear stress for columns having these mechanically reinforced shapes.

In other embodiments, the raised structures have improved mechanical stability and scratch resistance by having basal widths greater than distal widths. In some embodiments, these raised structures with improved stability and mechanical strength comprise posts, channels or closed-cell compartments. In some embodiments, the basal widths of the raised structures are greater than the distal widths by a factor of greater than 1:1 to greater than 10:1, or by a factor of between 2:1 and 9:1, between 3:1 and 8:1, between 4:1 and 7:1, or between 5:1 and 6:1. In other embodiments, the basal widths are 2 times, 3 times, 4 times, 5 times, 6 times, 7 times, 8 times, 9 times or 10 times wider than the distal widths. Such structures show 5-100 times higher mechanical stability and/or strength than their non-reinforced analogs, depending upon the ratio between the distal and basal widths. For example, FIG. 8D shows computer simulation results demonstrating that the force required to break exemplary conical structure is at least 10 times higher than that required to break cylindrical posts of the same distal width. As discussed above, FIG. 8A presents the stress field for a cylindrical Si column with the width of 1 μm, height 9 μm, which shows a maximum shear stress at the top of such structure of about 100 MPa. FIG. 8D presents the stress field for a conical Si column with the distal width of 1 μm, basal width of 2.7 μm, height 9 μm. The maximum shear stress at the top of such structure is about 1100 MPa, about an 11-fold improvement in maximum shear stress over a column lacking this mechanical reinforcement.

Under dynamic conditions, it is preferable to use a patterned surface displaying better mechanical robustness and droplet pressure stability. Indeed, a surface having mechanically reinforced raised structures, e.g., tapered compartments, displays improved mechanical stability, pressure stability, and/or superhydrophobicity/wetting transition. Note that the droplet pressure stability is related to the maximum pressure a droplet can exert on a patterned surface without transitioning to the wetted state.

Raised Post Structures

In some embodiments, the raised structures are highly aspected, such as rods, posts, or other structures having a widths smaller than the height. The shape of the posts can be cylindrical, pyramidal, conical, branched Y-, T-, X-, I-shaped, S-shaped in cross section or a combination thereof.

The raised structures in this embodiment typically have heights of 0.1 μm to 100 μm (preferably 1 μm to 25 μm and most preferably 2 μm to 10 μm).

For embodiments where the raised structures energetically exclude microorganisms from the substrate surface by anti-wetting properties under dynamic conditions, the raised structures have widths at their distal ends of 0.01 μm to 5 μm, and pitches of 0.05 μm to 50 μm (preferably 0.1 μm to 20 μm and most preferably 0.5 μm to 10 μm). Energetic exclusion by raised structures having these prescribed dimensions is demonstrated in Example 3 and FIG. 15A-C.

For embodiments where the raised structures physically exclude microorganisms from the substrate subsurface by controlling interstructure spacings and by limiting the available width for adhesion, and where the microorganisms are contacting only the top surface with reduced contact area, the raised structures have interstructure spacings of 0.01 μm to 10 μm (preferably 0.1 μm to 2 μm), and widths at their distal ends of 0.01 μm to 5 μm. More specifically, the physically-excluding surfaces should have interstructure spacings and structure widths that are smaller than the size of the microorganism contained in the contaminated solution or medium. These sizes should be tailored to the application and the specific species expected in the contaminated environment. Because the microorganisms are physically excluded from the subsurface, it is not required that the surface be hydrophobic. In some embodiments, the surface and raised structures are hydrophobic. In further embodiments, the surface and raised structures are superhydrophobic. In still further embodiments, the surface and raised structures are not hydrophobic.

In some embodiments, the widths of the raised structures are constant along their heights. In still further embodiments, the widths of the raised structures change along their heights. In some embodiments, the widths of the raised structures increase as they approach the basal surface from the distal ends. In some embodiments, the widths of the raised structures increase from top to bottom linearly, exponentially, or by some other gradient (e.g., having a cross-sectional profile which is curvilinear) as they approach the basal surface from the distal ends. In further embodiments, the widths of the raised structures increase in a step-wise fashion from the distal ends to the basal surface. In some embodiments, the profiles of the posts are either columnar, conical, pyramidal, prismatic or curvy.

The raised structures can be raised posts of a variety of shapes, including, but not limited to, circles, ellipses, or polygons (such as triangles, squares, pentagons, hexagons, octagons, and the like). Although the exemplary substrates described above illustrate raised posts having uniform shape and size, the shape and/or size of raised posts on a given substrate can vary. In particular embodiments, the raised structures are not randomly distributed. For example, the substrate can be an array of rows of raised posts, where the posts in a given row differ in size and/or shape from the posts in an adjacent row of raised posts. Alternatively, a first population of raised posts of similar size and/or shape can be disposed on the substrate at particular locations, and a second population of raised posts having different size and/or shapes from the first population can be disposed on the substrate at locations different from the first population, creating patterns of posts of different size and/or shape. The raised structures can also exhibit basal widths greater than distal widths. For example, basal widths can be greater than distal widths by ratios of greater than 1:1 to 10:1.

FIG. 3A shows a perspective-view schematic diagram of raised post structures. FIG. 4A shows top-view schematic diagrams of raised post structures having different shapes.

In some embodiments, the raised post structures described herein are structured to achieve improved stability and improved mechanical strength.

In some embodiments, reinforced post structures have shapes of branched I-, Y-, T- or X-columns or S-shaped in cross section. FIG. 5 A-D is a schematic representing cross-sectional views of reinforced branched I-shaped, T-shaped, X-shaped and Y-shaped raised post structures. FIG. 5E depicts scanning electron microscopy image of an array of exemplary array of branched T-shaped raised Si posts. FIG. 5F depicts light microscopy image of an array of raised branched Y-shaped polymeric post structures with improved mechanical strength fabricated using molding.

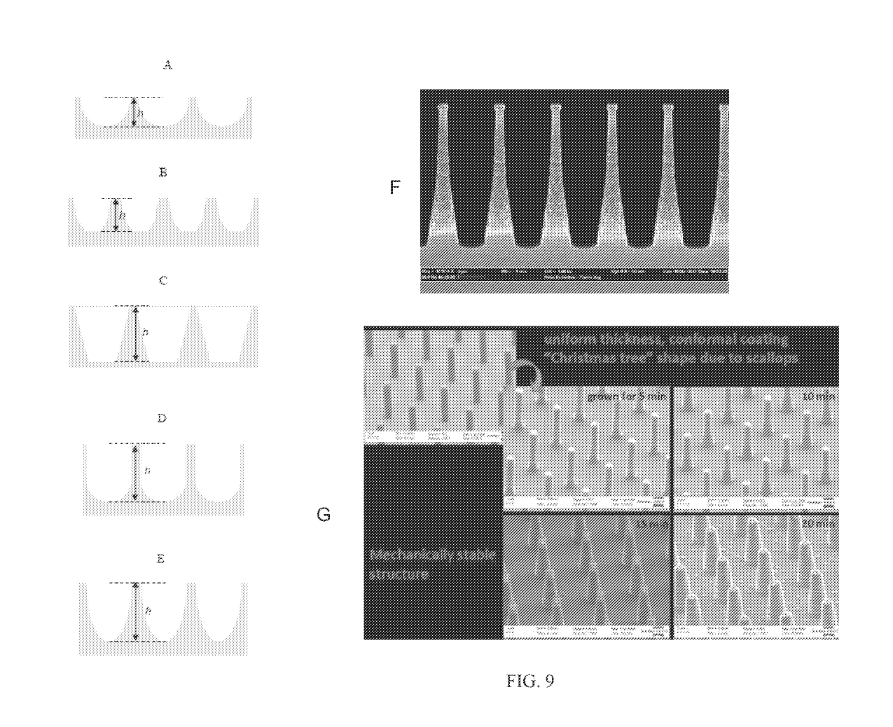

In some embodiments, raised post structures having basal widths greater than distal widths impart improved mechanical strength. FIG. 9A-E shows cross-sectional schematic diagrams of raised post structures having basal widths greater than distal widths] FIG. 9F depicts scanning electron microscopy image of an exemplary array of raised conical Si post structures with improved mechanical strength fabricated using Bosch process. FIG. 9G depicts scanning electron microscopy images of an array of raised conical polymeric post structures with improved mechanical strength fabricated using reshaping by electrodeposition of conducting polymers.

In some embodiments, the interstructure spacings of the raised structures are less than about the smallest axis of a microorganism. In further embodiments, the interstructure spacings of the raised structures are less than about the length and greater than about the transverse diameter of a microorganism. In still further embodiments, interstructure spacings of the raised structures are greater than the about the largest axis of a microorganism. In further embodiments, as the interstructure spacings of the raised structures decrease and are less than about the shortest dimension of a microorganism, the microorganism contacts the tips of the structures and does not contact the substrate.

In some embodiments, the raised post structures described herein are applied as a coating to a substrate to imbue the substrate with the desired antibiofilm properties.

Raised Channel Structures

In some embodiments, the raised structures define a plurality of lateral walls, creating channel structures, grooves or blades, which can be sinuous. The term “groove” refers to a channel that is delimited by a bottom surface and two raised continuous structures, e.g., two non-intersecting walls.

In some embodiments, the raised structures define lateral walls that are substantially straight and parallel along their entire length. In further embodiments, the raised structures define lateral walls that are curved, jagged, or have other reinforced geometries and arrangements (e.g., sinusoidal, wavy or zigzag), maintaining the interstructure spacings described below. Although the exemplary substrates describe raised structures defining lateral walls of uniform shape and size, the shape and/or size of the lateral walls on a given substrate can vary.

FIG. 3B shows a perspective-view schematic diagram of raised channel structures. FIG. 4B shows a top-view schematic diagram of various raised channel structures having straight, curvy and random shapes.

The raised structures in this embodiment typically have heights of 0.1 μm to 100 μm (preferably 1 μm to 25 μm and most preferably 2 μm to 10 μm).

For embodiments where the raised structures energetically exclude microorganisms from the substrate surface by anti-wetting properties under dynamic conditions, the raised structures have widths at their distal ends of 0.01 μm to 5 μm, and pitches of 0.05 μm to 50 μm (preferably 0.2 μm to 20 μm and most preferably 0.5 μm to 10 μm).

For embodiments where the raised structures physically exclude microorganisms from the substrate subsurface by controlling interstructure spacings, the raised structures have interstructure spacings of 0.01 μm to 10 μm (preferably 0.1 μm to 2 μm), and widths at their distal ends of 0.01 μm to 5 μm. More specifically, the physically-excluding surfaces should have interstructure spacings and structure widths that are smaller than the size of the microorganism contained in the contaminated solution or medium. These sizes should be tailored to the application and the specific species expected in the contaminated environment. Because the microorganisms are physically excluded from the subsurface, it is not required that the surface be hydrophobic. In some embodiments, the surface and raised structures are hydrophobic. In further embodiments, the surface and raised structures are superhydrophobic. In still further embodiments, the surface and raised structures are not hydrophobic.

In some embodiments, the widths of the raised structures are constant along their heights (e.g., defining flat-bottomed channels as shown in FIG. 3B). In still further embodiments, the widths of the raised structures change along their heights. In some embodiments, the channels are defined by raised structures whose widths increase as they approach the basal surface from the distal ends. In some embodiments, the widths of the raised structures increase linearly, exponentially, or by some other gradient (e.g., having a cross-sectional profile which is curvilinear, defining round-bottomed channels) as they approach the basal surface from the distal ends. In further embodiments, the widths of the raised structures increase in a step-wise fashion from the distal ends to the basal surface.

In some embodiments, the interstructure spacings of the raised structures are less than about the smallest axis of a microorganism. In further embodiments, the interstructure spacings of the raised structures are less than about the length and greater than about the transverse diameter of a microorganism. In still further embodiments, interstructure spacings of the raised structures are greater than the about the largest axis of a microorganism. In further embodiments, as the interstructure spacings of the raised structures decrease and are less than about the shortest dimension of a microorganism, the microorganism contacts the tips of the structures and does not contact the substrate.

In some embodiments, the raised post structures described herein are modified to achieve improved stability and improved mechanical strength. In some embodiments, raised channel structures having basal widths greater than distal widths impart improved mechanical strength.

In some embodiments, the raised channel structures described herein are applied as a coating to a substrate to imbue the substrate with the desired antibiofilm properties.

Raised Closed-Cell Structures

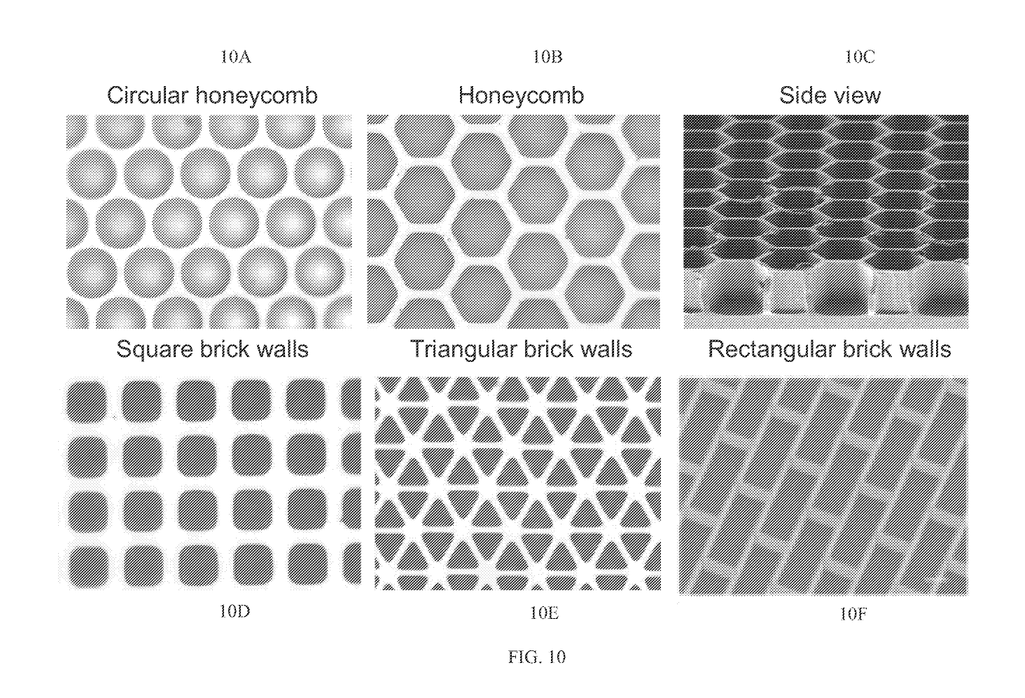

In some embodiments, the raised structures are interconnected walls that form closed-cell structures or compartments, i.e., cavities each delimited by a bottom surface and one or more walls. A closed-cell structure includes a plurality of walls that define an enclosed space. In some embodiments, the closed-cell structures share walls with adjacent closed-cells and form a closely packed array of closed-cell structures (see FIG. 3C and FIGS. 10A-10F). Such closed-cell structures with interconnected walls have much improved mechanical properties and scratch resistance compared with post or channels structures.

The raised structures in this embodiment typically have heights of 0.1 μm to 100 μm (preferably 1 μm to 25 μm and most preferably 2 μm to 10 μm).

For embodiments where the raised structures energetically exclude microorganisms from the substrate surface by anti-wetting properties under dynamic conditions, the raised structures have widths at their distal ends of 0.01 μm to 5 μm, and shortest wall-to-wall distances within each compartment of 0.02 μm to 50 μm (preferably 0.2 μm to 20 μm and most preferably 0.5 μm to 10 μm).

For embodiments where the raised structures physically exclude microorganisms from the substrate subsurface by controlling interstructure spacings, the raised structures have interstructure spacings of 0.01 μm to 10 μm (preferably 0.1 μm to 2 μm), and widths at their distal ends of 0.01 μm to 5 μm. More specifically, the physically-excluding surfaces should have interstructure spacings and structure widths that are smaller than the size of the microorganism contained in the contaminated solution or medium. These sizes should be tailored to the application and the specific species expected in the contaminated environment. Because the microorganisms are physically excluded from the subsurface, it is not required that the surface be hydrophobic. In some embodiments, the surface and raised structures are hydrophobic. In further embodiments, the surface and raised structures are superhydrophobic. In still further embodiments, the surface and raised structures are not hydrophobic.

In some embodiments, the closed-cell structures are defined by raised structures having widths which are constant along their heights (e.g., defining flat-bottomed compartments). In still further embodiments, the closed-cell structures are defined by raised structures having widths which change along their heights. In some embodiments, the closed-cell structures are defined by raised structures whose widths increase as they approach the basal surface from the distal ends. In some embodiments, the widths of the raised structures increase linearly, exponentially, or by some other gradient (e.g., having a cross-sectional profile which is curvilinear, defining round-bottomed compartments) as they approach the basal surface from the distal ends. In further embodiments, the widths of the raised structures increase in a step-wise fashion from the distal ends to the basal surface.

Based on the number of interconnected raised structures and the angle between two consecutive raised structures, compartments of different geometries can be formed. Examples of such compartments include, but are not limited to, square compartments (i.e. delimited by four identical walls), rectangular compartments (i.e., delimited by four walls and each two opposite walls are identical), triangular compartments (i.e., delimited by three walls), hexagonal compartments (i.e., delimited by six walls), circular or elliptical compartments (i.e., delimited by one wall), randomly-shaped compartments, and combinations thereof. Other raised structures can include any other raised structures, such as an array of closed-cell structures, an array of honeycombs, an array of egg closed walls, an array of bricks, and the like. In some embodiments, the compartments are regularly shaped. In further embodiments, the compartments are irregularly shaped. For example, the closed-cell structures can resemble a web pattern, with the closed-cells varying in shape and dimension. In other examples, the substrate contains pores of varying size and shape.

FIG. 3C shows a perspective-view schematic diagram of raised closed-cell brick structures. FIG. 4C shows a top-view schematic diagram of raised closed-cell structures which do not share walls, and are spaced apart from one another. FIG. 4D shows a top-view schematic diagram of raised closed-cell brick, square, honeycomb and web structures. FIG. 10 shows optical and electron micrographs of exemplary raised closed-cell structures comprising honeycombs and brick walls.

The pattern formed by the raised structures and the compartments may vary based on the spatial arrangement of the raised structures (i.e., walls). In some embodiments, the raised closed-cell structures share walls (see, e.g., FIG. 4D). For example, parallel longitudinal walls intersecting with transverse (e.g., perpendicular) walls form rows of parallel compartments, e.g., “brick-like” compartments. Compartments in two adjacent and parallel rows can be staggered. In some embodiments, these closed-cell structures display improved mechanical stability and scratch resistance. In further embodiments, the raised closed-cell structures do not have intersecting walls (see, e.g., FIG. 4C).

In some embodiments, the raised closed-cell structures described herein are modified further to achieve improved stability and improved mechanical strength. In some embodiments, raised closed-cell structures having basal widths greater than distal widths impart improved mechanical strength.

In some embodiments, the raised closed-cell structures described herein are applied as a coating to a substrate to imbue the substrate with the desired antibiofilm properties.

A substrate for use in this invention can have one or more of the above-described surface patterns.

Methods of Making

The raised structures of the present invention can be produced by any known method for depositing raised structures onto substrates. Nonlimiting examples include conventional photolithography, projection lithography, e-beam writing or lithography, depositing nanowire arrays, growing nanostructures on the surface of a substrate, soft lithography, replica molding, solution deposition, solution polymerization, electropolymerization, electrospinning, electroplating, vapor deposition, contact printing, etching, transfer patterning, microimprinting, self-assembly, and the like. For example, a silicon substrate having a post array, a brick array, a channel or “blade” array, a box array, or a honeycomb array can be fabricated by photolithography using the Bosch reactive ion etching method (as described in Plasma Etching: Fundamentals and Applications, M. Sugawara, et. al, Oxford University Press, (1998), ISBN-10: 019856287X), hereby incorporated by reference in its entirety. Further exemplary methods are described in WO 2009/158631, hereby incorporated by reference in its entirety

Patterned surfaces can also be obtained as replicas (e.g., epoxy replicas) by a soft lithographic method (see, e.g., Pokroy et al., Advanced Materials, 2009, 21, 463, hereby incorporated by reference in its entirety [Patterned surfaces having round-bottoms (e.g., a round-bottomed brick array) can be obtained by a combination of the Bosch reactive ion etching method and the isotropic reactive etching technique described in Plasma Etching: Fundamentals and Applications, M. Sugawara, et. al., Oxford University Press, (1998), ISBN-10: 019856287X, hereby incorporated by reference in its entirety.

Polymer films with patterned surfaces can be fabricated by means known in the art (e.g., roll-to-roll imprinting or embossing).

A patterned surface thus formed, if not fabricated from an innately hydrophobic material, can be coated with a hydrophobic material, such as low-surface-energy fluoropolymers (e.g., polytetrafluoroethylene), and fluorosilanes (e.g., heptadecylfluoro-1,1,2,2-tetra-hydrodecyl-trichlorosilane). Surface coating can be achieved by methods well known in the art, including plasma assisted chemical vapor deposition, solution deposition, and vapor deposition.

Note that the patterned surface can either be an integral part of the substrate or a separate layer on the substrate. For example, a patterned surface can be fabricated from a material (e.g., a silicon wafer or a polymer film) and used to cover another material (e.g., an aluminum plate). This can be useful when it is easier to fabricate a patterned surface from a material other than that of the substrate. Also, to obtain a large patterned surface on a large substrate, it is often necessary to fabricate smaller patterned surfaces and then place them on the large substrate.

To cover a substrate with a patterned surface, one can use standard methods (e.g., tiling, embossing, and rolling with a patterned roller, etc), as described in Whitesides et al., Chem. Review, 2005, 105, 1171-1196, hereby incorporated by reference in its entirety. To analyze the topology of a patterned surface, one can use well-known methods, such as scanning electron microscopy (SEM) and atomic force microscopy (AFM). As mentioned above, a water droplet on a hydrophobic surface for use in this invention displays a contact angle of more than 90°, preferably more than 140°. The actual contact angle can be determined by methods well known in the art (e.g., with a contact angle goniometer).

The raised structures described herein can also be fabricated using molding techniques, such as those described in WO 2009/158631, published Dec. 30, 2009, hereby incorporated by reference in its entirety. These techniques involve making an original replica mold using any known techniques, followed by forming a negative replica mold using suitable replica material. Finally, a replica is made using the negative replica as a mold. These replicas can then coat any flat or curved surface (including the inner or outer side of pipes as shown in Such curved patterned tubes are of particular importance in applications related to catheters or vascular tubing.

The raised structures described herein can also be fabricated using electrodeposition techniques, such as those described in U.S. Pat. Application No. 61/365,615, filed Jul. 19, 2010, hereby incorporated by reference in its entirety. In particular, the raised structures described herein can be fabricated by in situ deposition of conducting organic polymers by either electrochemical deposition or electroless direct solution deposition. In these methods, the morphology of the conducting organic polymers can be controlled by varying the deposition conditions such as the concentration of monomer, the types of electrolytes and buffers, the deposition temperature and time, and the electrochemical conditions such as voltage and current. The morphology of conducting organic polymers can be finely controlled from nanometer to over micrometer scales. Therefore, surface coatings with precisely controlled morphology can be produced by simple modifications, which promise the customization of various surface properties by design and control of the morphology.

The raised structures described herein can be made of any suitable material. Nonlimiting examples of such materials include polymers such as epoxy, polypropylene (PP), polyethylene (PE), polyvinylalcohol (PVA), poly methyl methacrylic acid (PMMA), and various hydrogels and biological macromolecules (e.g., alginates, collagen, agar); metals and alloys, such as Au metal and Ti alloys; and ceramics including Al₂O₃, TiO₂, HfO₂, SiO₂, ZrO, and BaTiO₃. Other polymeric materials, metals, alloys and ceramics can also be used.

In some embodiments, the material is any biocompatible material capable of being formed into a raised structure described herein.

### Hydrophobic Coatings

In some embodiments, after fabrication, the raised structures are then treated with a hydrophobic coating to render the raised structures superhydrophobic. For example, as discussed above, hydrophobic surface coatings can be applied using fluorinated silanes, either by solution or vapor deposition treatment.

In some embodiments, the raised structures are rendered superhydrophobic by treatment with a silicone fluid, such as a polysiloxane, an alkyl silane, or an alkyl silazane. Nonlimiting examples of suitable polysiloxanes include a linear, branched or cyclic polydimethylsiloxane; polysiloxanes having a hydroxyl group in the molecular chain such as silanol-terminated polydimethylsiloxane, silanol-terminated polydiphenylsiloxane, diphenylsilanol-terminated polydimethylphenylsiloxane, carbinol-terminated polydimethylsiloxane, hydroxypropyl-terminated polydimethylsiloxane and polydimethyl-hydroxyalkylene oxide methylsiloxane; polysiloxanes having an amino group in the molecular chain such as bis(aminopropyldimethyl)siloxane, aminopropyl-terminated polydimethylsiloxane, aminoalkyl group-containing, T-structured polydimethylsiloxane, dimethylamino-terminated polydimethylsiloxane and bis(aminopropyldimethyl)siloxane; polysiloxanes having a glycidoxyalkyl group in the molecular chain such as glycidoxypropyl-terminated polydimethylsiloxane, glycidoxypropyl-containing, T-structured polydimethylsiloxane, polyglycidoxypropylmethylsiloxane and a polyglycidoxypropylmethyldimethylsiloxane copolymer; polysiloxanes having a chlorine atom in the molecular chain such as chloromethyl-terminated polydimethylsiloxane, chloropropyl-terminated polydimethylsiloxane, polydimethyl-chloropropylmethylsiloxane, chloro-terminated polydimethylsiloxane and 1,3-bis(chloromethyl)tetramethyldisiloxane; polysiloxanes having a methacryloxyalkyl group in the molecular chain such as methacryloxypropyl-terminated polydimethylsiloxane, methacryloxypropyl-containing, T-structured polydimethylsiloxane and polydimethyl-methacryloxypropylmethylsiloxane; polysiloxanes having a mercaptoalkyl group in the molecular chain such as mercaptopropyl-terminated polydimethylsiloxane, polymercaptopropylmethylsiloxane and mercaptopropyl-containing, T-structured polydimethylsiloxane; polysiloxanes having an alkoxy group in the molecular chain such as ethoxy-terminated polydimethylsiloxane, polydimethylsiloxane having trimethoxysilyl on one terminal and a polydimethyloctyloxymethylsiloxane copolymer; polysiloxanes having a carboxyalkyl group in the molecular chain such as carboxylpropyl-terminated polydimethylsiloxane, carboxylpropyl-containing, T-structured polydimethylsiloxane and carboxylpropyl-terminated, T-structured polydimethylsiloxane; polysiloxanes having a vinyl group in the molecular chain such as vinyl-terminated polydimethylsiloxane, tetramethyldivinyldisiloxane, methylphenylvinyl-terminated polydimethylsiloxane, a vinyl-terminated polydimethyl-polyphenylsiloxane copolymer, a vinyl-terminated polydimethyl-polydiphenylsiloxane copolymer, a polydimethyl-polymethylvinylsiloxane copolymer, methyldivinyl-terminated polydimethylsiloxane, a vinyl terminated polydimethylmethylvinylsiloxane copolymer, vinyl-containing, T-structured polydimethylsiloxane, vinyl-terminated polymethylphenetylsiloxane and cyclic vinylmethylsiloxane; polysiloxanes having a phenyl group in the molecular chain such as a polydimethyl-diphenylsiloxane copolymer, a polydimethyl-phenylmethylsiloxane copolymer, polymethylphenylsiloxane, a polymethylphenyl-diphenylsiloxane copolymer, a polydimethylsiloxane-trimethylsiloxane copolymer, a polydimethyl-tetrachlorophenylsiloxane copolymer and tetraphenyldimethylsiloxane; polysiloxanes having a cyanoalkyl group in the molecular chain such as polybis(cyanopropyl)siloxane, polycyanopropylmethylsiloxane, a polycyanopropyl-dimethylsiloxane copolymer and a polycyanopropylmethyl-methyphenylsiloxane copolymer; polysiloxanes having a long-chain alkyl group in the molecular chain such as polymethylethylsiloxane, polymethyloctylsiloxane, polymethyloctadecylsiloxane, a polymethyldecyl-diphenylsiloxane copolymer and a polymethylphenetylsiloxane-methylhexylsiloxane copolymer; polysiloxanes having a fluoroalkyl group in the molecular chain such as polymethyl-3,3,3-trifluoropropylsiloxane and polymethyl-1,1,2,2-tetrahydrofluorooctylsiloxane; polysiloxanes having a hydrogen atom in the molecular chain such as hydrogen-terminated polydimethylsiloxane, polymethylhydrosiloxane and tetramethyldisiloxane; hexamethyldisiloxane; and a polydimethylsiloxane-alkylene oxide copolymer. Many polysiloxanes are commercially available as water repellents, such as Super Rain X formed mainly of polydimethylsiloxane (supplied by Unelko) and Glass Clad 6C formed mainly of polydimethylsiloxane whose terminal groups are replaced with chlorine atom (supplied by Petrarch Systems Inc.). These polysiloxanes can be used alone or in combination. Other suitable polysiloxanes are those organic polysiloxanes disclosed in U.S. Pat. No. 5,939,491, which is hereby incorporated by reference in its entirety.

Suitable alkyl silanes include, but are not limited to, n-butyltrimethoxysilane, n-decyltrimethoxysilane, isobutyltrimethoxysilane, n-hexyltrimethoxysilane, and cyclohexylmethyldimethoxysilane. Alkyl silanes can be used separately or in a mixture of two or more. Alternatively, a fluorinated hydrophobic silane can be used such as perfluorinated alkyl, ether, ester, urethane, or other chemical moiety possessing fluorine and a hydrolyzable silane. Other exemplary fluorosilanes that can be used to coat raised structures are described in U.S. Pat. Nos. 5,081,192; 5,763,061; and 6,227,485, hereby incorporated by reference in their entireties.

The raised structures can be totally coated or partially coated, such as the vertical end of the raised structure opposite the substrate. In some embodiments, the raised nanostructures and the substrate are coated with the hydrophobic coating. The coating can be applied at a thickness of about 1 nm to about 30 nm.

If the structures are made out of hydrophobic material, no additional hydrophobic coating is required.

The superhydrophobicity can be quantified by measuring the contact angle between a droplet of a contaminated liquid and the surface of an array of raised superhydrophobic structures using known methods. In particular embodiments, the array has a contact angle of greater than about 140°, or greater than about 150°, or greater than about 155° or greater than about 160°, or greater than about 165° or greater than about 170°, or greater than about 175°.

### Microorganisms

### Bacterial Cells

In certain embodiments, the raised structures described herein can be used to prevent, inhibit, or reduce the attachment of bacteria onto a substrate. In exemplary methods, the bacteria are biofilm-forming bacteria. The bacteria may be a gram negative bacteria species or a gram positive bacteria species. Nonlimiting examples of such bacteria include a member of the genus Actinobacillus (such as Actinobacillus actinomycetemcomitans), a member of the genus Acinetobacter (such as Acinetobacter baumannii), a member of the genus Aeromonas, a member of the genus Bordetella (such as Bordetella pertussis, Bordetella bronchiseptica, or Bordetella parapertussis), a member of the genus Brevibacillus, a member of the genus Brucella, a member of the genus Bacteroides (such as Bacteroides fragilis), a member of the genus Burkholderia (such as Burkholderia cepacia or Burkholderia pseudomallei), a member of the genus Borelia (such as Borelia burgdorfen), a member of the genus Bacillus (such as Bacillus anthracis or Bacillus subtilis), a member of the genus Campylobacter (such as Campylobacter jejuni), a member of the genus Capnocytophaga, a member of the genus Cardiobacterium (such as Cardiobacterium hominis), a member of the genus Citrobacter, a member of the genus Clostridium (such as Clostridium tetani or Clostridium difficile), a member of the genus Chlamydia (such as Chlamydia trachomatis, Chlamydia pneumoniae, or Chlamydia psiffaci), a member of the genus Eikenella (such as Eikenella corrodens), a member of the genus Enterobacter, a member of the genus Escherichia (such as Escherichia coli), a member of the genus Entembacter, a member of the genus Francisella (such as Francisella tularensis), a member of the genus Fusobacterium, a member of the genus Flavobacterium, a member of the genus Haemophilus (such as Haemophilus ducreyi or Haemophilus influenzae), a member of the genus Helicobacter (such as Helicobacter pylori), a member of the genus Kingella (such as Kingella kingae), a member of the genus Klebsiella (such as Klebsiella pneumoniae), a member of the genus Legionella (such as Legionella pneumophila), a member of the genus Listeria (such as Listeria monocytogenes), a member of the genus Leptospirae, a member of the genus Moraxella (such as Moraxella catarrhalis), a member of the genus Morganella, a member of the genus Mycoplasma (such as Mycoplasma hominis or Mycoplasma pneumoniae), a member of the genus Mycobacterium (such as Mycobacterium tuberculosis or Mycobacterium leprae), a member of the genus Neisseria (such as Neisseria gonorrhoeae or Neisseria meningitidis), a member of the genus Pasteurella (such as Pasteurella multocida), a member of the genus Proteus (such as Proteus vulgaris or Proteus mirablis), a member of the genus Prevotella, a member of the genus Plesiomonas (such as Plesiomonas shigelloides), a member of the genus Pseudomonas (such as Pseudomonas aeruginosa), a member of the genus Providencia, a member of the genus Rickettsia (such as Rickettsia rickettsii or Rickettsia typhi), a member of the genus Stenotrophomonas (such as Stenotrophomonas maltophila), a member of the genus Staphylococcus (such as Staphylococcus aureus or Staphylococcus epidermidis), a member of the genus Streptococcus (such as Streptococcus viridans, Streptococcus pyogenes (group A), Streptococcus agalactiae (group B), Streptococcus bovis, or Streptococcus pneumoniae), a member of the genus Streptomyces (such as Streptomyces hygroscopicus), a member of the genus Salmonella (such as Salmonella enteriditis, Salmonella typhi, or Salmonella typhimurium), a member of the genus Serratia (such as Serratia marcescens), a member of the genus Shigella, a member of the genus Spirillum (such as Spirillum minus), a member of the genus Treponema (such as Treponema pallidum), a member of the genus Veillonella, a member of the genus Vibrio (such as Vibrio cholerae, Vibrio parahaemolyticus, or Vibrio vulnificus), a member of the genus Yersinia (such as Yersinia enterocolitica, Yersinia pestis, or Yersinia pseudotuberculosis), and a member of the genus Xanthomonas (such as Xanthomonas maltophilia).

### Fungal Cells

In some embodiments, the raised structures described herein can be used to prevent, inhibit, or reduce the attachment of fungi onto a substrate. In exemplary methods, the fungi are biofilm-forming fungi. Fungal species that can be controlled using the methods described herein include, but are not limited to, a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

### Viral Cells

In some embodiments, the raised structures described herein can be used to prevent, inhibit, or reduce the attachment of viruses onto a substrate. Viral species that can be controlled using the methods described herein include, but are not limited to, Cytomegalovirus (CMV), dengue, Epstein-Barr, Hantavirus, Human T-cell lymphotropic virus (HTLV I/II), Parvovirus, Hepatitis A, B, or C, human papillomavirus (HPV), respiratory syncytial virus (RSV), Varicella zoster, West Nile, herpes, polio, smallpox, and yellow fever.

### Using Raised Structures

Substrates having raised structures described herein can be used to inhibit or reduce the attachment of a microorganism to the substrate. Such a surface can be any surface, preferably hard surfaces, which may be prone to adhesion of microorganisms. Examples of contemplated surfaces include hard surfaces made from one or more of the following materials: metal, plastic, rubber, board, glass, wood, paper, concrete, rock, marble, gypsum and ceramic materials, such as porcelain, which optionally are coated, for example, with paint or enamel.

Substrates can be treated with a raised feature using replica molding to produce surfaces with high aspect ratio raised features. Replica molding can be used for form sheets that can be applied to an article surface, for example, using glue or other adhesive. Replica molding can also be used to form and object directly having the raised feature treated surface. Further detail on suitable replica molding techniques are described in WO 2009/158631, which is hereby incorporated by reference in its entirety

In certain embodiments, the surface is a medical device, instrument, or implant. Nonlimiting examples include clamps, forceps, scissors, skin hooks, tubing (such as endotracheal or gastrointestinal tubes), needles, retractors, scalers, drills, chisels, rasps, saws, catheters including indwelling catheter (such as urinary catheters, vascular catheters, peritoneal dialysis catheter, central venous catheters), catheter components (such as needles, Leur-Lok connectors, needleless connectors), orthopedic devices, artificial heart valves, prosthetic joints, voice prostheses, stents, shunts, pacemakers, surgical pins, respirators, ventilators, and endoscopes. In one or more embodiments, raised structures are prepared and are attached to a device, such as a medical device. In other embodiments, the raised structures are molded directly into the device structure, or imprinted on the device surface.

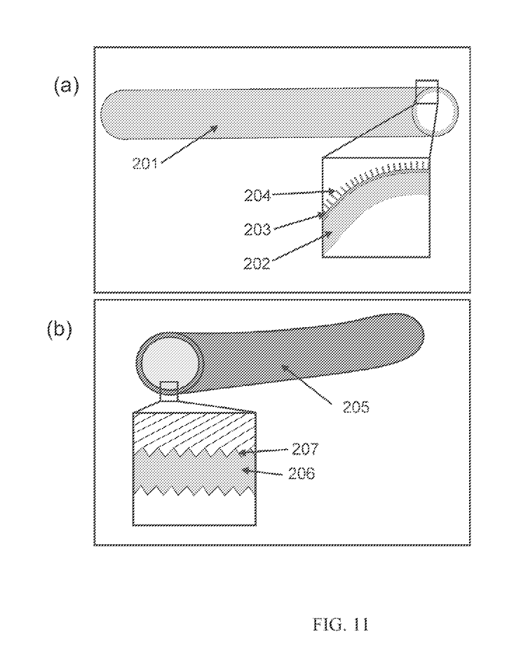

FIG. 11 is an illustration of one or more embodiments of the invention. FIG. 11A depicts a perspective view of a portion of a medical device 201 which has a surface 202 coated by a surface coating 203 exhibiting raised structures 204. FIG. 11B depicts a perspective view of a portion of a medical device 205 with a surface 206 which comprises raised structures 207. In FIG. 2B, the device is not coated with a surface coating, but rather the surface itself bears the raised structures described herein. As discussed above, the raised structures are constructed so as to imbue the device with antimicrobial properties.

Other substrates include surfaces of drains, tubs, kitchen appliances, countertops, shower curtains, grout, toilets, industrial food and beverage production facilities, and flooring. Other surfaces include marine structures, such as boats, piers, oil platforms, water intake ports, sieves, and viewing ports.

In particular applications, raised superhydrophobic structures can be applied to medical devices, such as surgical instruments or catheters, that are inserted into the body, to prevent the contamination of such devices upon splashing or exposure to contaminated solutions in the external environment (e.g., prior to insertion). Such surface treatments may be particularly important in emergency medical situations, including military environments, where the control of sterility and cleanliness is not easily achieved and the medical instruments or implant surfaces are exposed to, splashed by or washed with contaminated liquids.

The invention is further described in the following examples, which do not limit the scope of the invention described in the claims.

### EXAMPLES

### Example 1

### Fabrication of Patterned Hydrophobic Surfaces

Photolithography following the Bosch process was used to fabricate from 100 mm silicon wafers numerous surfaces of different patterns, including a cylindrical post array, a honeycomb array, a brick array, a box array, and a channel array. The table below lists 5 different fabricated patterned surfaces with the given dimensions. It also lists water contact angles for certain surfaces coated with a fluorinated compound, as described below.

TABLE 1

| | | Width | Pitch/Size | | Contact |
|---|---|---|---|---|---|
| Type | Name | (μm) | (μm) | Depth (μm) | Angle (°) |
| Post | | 1.5 | 3.6 | 8 | |
| Post | | 1.5 | 8 | 8 | |
| Post | | 2.0 | 10 | 10 | |
| Post | | 1.8 | 12 | 7 | |
| Post | | 1.5 | 16 | 9 | |
| Post | Post2-F | 0.3 | 2 | 10 | 171 |
| Post | Post5-F | 1.5 | 3.5 | 10 | 162 |
| Honeycomb | | 3.5 | 40 | 15 | |
| Brick | Brick40-F | 1.3 | 16 × 40 | 18 | 149 |
| Box | | 1.4 | 100 × 200 | 10 | |
| Box | | 1.4 | 100 × 800 | 10 | |
| Box | | 1.4 | 200 × 400 | 10 | |
| Channel | | 1 | 5 | | |

The patterns were created by contact printing using 0.5 μm thick S1805 positive photoresist. Separate contact masks were fabricated to print a 60×60 or 40×40 mm square on silicon wafers. The patterns were then etched into the silicon wafers using the Bosch process, which uses two separate steps to create vertical sidewalls. Thus, SF₆ was first used to etch the Si, and then C₄F₈ was used to deposit a protective layer of fluoropolymer to prevent further Si etching. Vertical sidewalls were formed with certain undercuts and ripples relative to the mask. The photoresist was then stripped using oxygen plasma, and the wafers were cleaned with H₂SO₄/H₂O₂ Piranha wet etch. For surfaces with submicron structures, projection lithography was used instead of contact lithography.

Epoxy (i.e., non-silicon) patterned substrates were also fabricated by replication of the silicon masters following the soft lithographic method described in Pokroy et al., Advanced Materials, 2009, 21, 463, hereby incorporated by reference in its entirety.

To form a hydrophobic surface, each patterned surface was coated with a thin layer (approximately 2 nm) of a fluorinated compound (e.g., heptadecylfluoro-1,1,2,2-tetra-hydrodecyl-trichlorosilane) using plasma assisted chemical vapor deposition. More specifically, the fluorinated compound was deposited from vapor on the surface in a vacuum chamber at 25° C. for 10 h.

All fabricated patterned surfaces were analyzed by SEM and the contact angle of a water droplet on certain patterned surface was determined by a standard goniometer with a high resolution camera designed for the measurements of contact angles.

SEM photomicrographs of silicon posts, honeycombs, and bricks similar to those used for the method of this invention and further details on the preparation of patterned hydrophobic surfaces can be found in Krupenkin et al., Langmuir, 2004, 20, 3824-3827, Henoch et al., AIAA Paper, 2006-3192, San Francisco, Calif., June 2006, and Ahuja et al., Langmuir, 2008, 24, 9-14, hereby incorporated by reference in their entireties.

### Example 2

### Fabrication of Raised Structures Using Electrodeposition

Pyrrole (Py) was purified by an alumina column prior to use. An aqueous solution of 0.08-0.14 M pyrrole in phosphate buffered saline (PBS) buffer and with 0.07 M lithium perchlorate (LiClO₄) was used for electrodeposition of PPy. Typical three electrode configuration was used with a Pt wire and mesh counter electrode and a Ag/AgCl reference electrode. Linear scanning voltammetry starting from 0-0.5 V to 0.8-1.0 V at a rate of 1 mV/s was typically applied to the sample surface as working electrode for growth of thin PPy film followed by chronoamperometry at about 0.85 V for additional time to grow fibrous PPy. For the continuous film deposition, an aqueous solution of 0.1 M pyrrole and 0.1 M sodium dodecylbenzene sulfonate (Na⁺DBS⁻) was prepared and purged by dry nitrogen for 10 minutes. To this solution, a template structure with patterned metal electrodes, as a working electrode, was placed then the polypyrrole films were electrochemically deposited using a standard three electrode configuration. An anodic potential of +0.55 V vs. Ag/AgCl (saturated with NaCl) was applied under a potentiostatic condition and a platinum mesh was used as a counter electrode. A gradient of the thickness of the deposited polypyrrole film was created by withdrawing the sample at a constant rate from the solution over a total deposition time. Freshly deposited polypyrrole layer was washed with deionized water and air blow dried.

The raised structures can be designed such that they exhibit improved mechanical strength against impact and scratch. An example of reinforced raised structures of post arrays is shown in FIG. 9G. The diameter of the basal part of each micropost was increased by depositing PPy of varying thickness. In this particular example, the metal electrodes were deposited by line-of-sight evaporation from an evaporation source aligned along the direction of each micropost. Due to the presence of scalloping (sidewall corrugation), the electrodes on the sidewall of each post form a series of isolated rings. As the electrodeposition of PPy takes place from the bottom surface, these isolated ring electrodes are electrically bridged by the freshly deposited, conductive PPy film. As a result, the basal part has thicker PPy layer than the top part and transforms a cylindrical post to a conical post reinforcing its mechanical properties.

### Example 3

### Growth of Pseudomonas aeruginosa on Raised Post and Closed-Cell Structures in Energetic Exclusion Experiments

A series of demonstration experiments were performed to test the effectiveness of various superhydrophobic surfaces to remain sterile after being exposed to a bacterial growth medium solution. Surfaces bearing raised post array structures of etched Si having posts of 5 lam and 1.5 μm widths, and epoxy (cast from a Si original) having posts of 300 nm widths were used as test samples as shown in FIG. 2. Each surface having raised post structures was treated with a hydrophobic silane ((heptadecafluoro-1,1,2,2-tetrahydrodecyl)trichlorosilane, Gelest) after oxygen plasma treatment, and was superhydrophobic (FIG. 2C-F). Two flat (unstructured) control samples were also used in comparison; clean, hydrophilic Si (Si—C) and fluorinated, hydrophobic Si (Si—F) (FIGS. 2A and 2B, respectively).

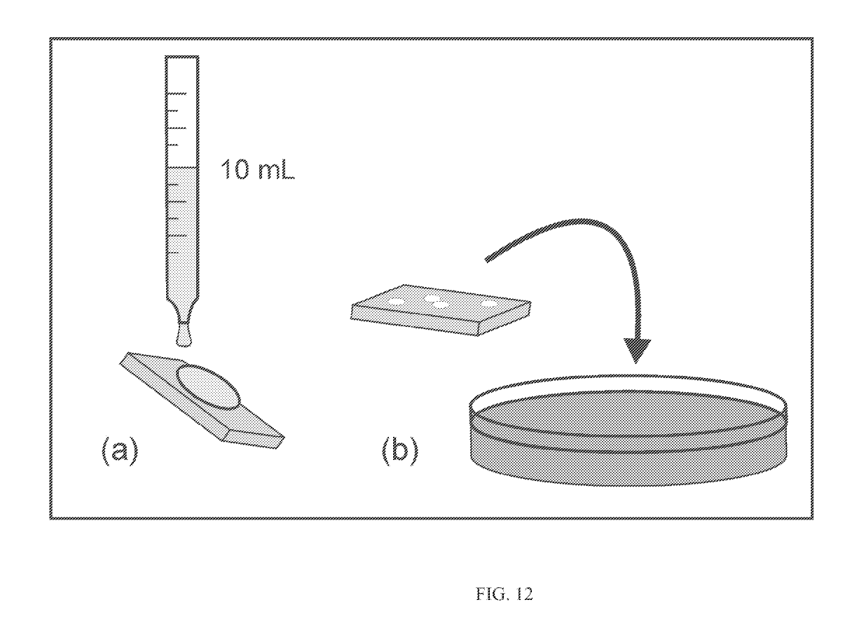

Each sample was exposed to a 10 mL flow of Pseudomonas aeruginosa, grown in TB medium for a period of 12 h (37° C., shaker) to an optical density (OD) of 0.2, after culture in a streaked TB-agar plate. The bacterial medium was exposed to the samples as a continuous stream from a 10 mL burette over about 5-7 s, as illustrated in FIG. 12A. The exposed samples were then immediately rinsed with PBS solution, and then placed, exposed side down, onto a fresh agar plate for a period of 10 min as illustrated in FIG. 12B. Each sample was then removed from the agar plates, and the plates were left for a period of 12 h, either at room temperature or at 37° C.

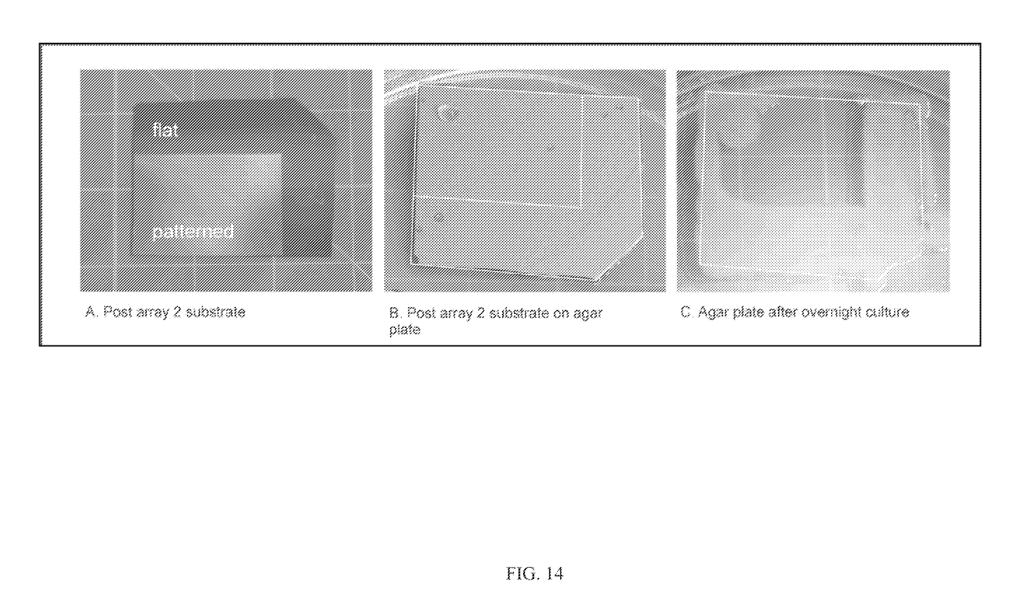

Qualitatively, the results of the agar plates are shown in FIG. 13-16. FIG. 13A-B shows that the Si—C and Si—F control samples produced regions of very obvious bacterial colonies in the contaminated agar plate (37° C.), and particularly so for the Si—C (hydrophilic) sample. FIG. 14 depicts an image of a substrate having both unpatterned (flat) and patterned raised post array surfaces, which after splashing of contaminated liquid and overnight exposure to the agar plate, shows that an area corresponding to the patterned surface is substantially free of microorganisms, while the area corresponding to the flat surface has significant microbial growth.

FIG. 15 depicts bacterial growth experiments after the exposure to the flow of contaminated liquid, as a function of the width of the raised posts. FIG. 15A depicts an image of a substrate bearing raised structures of posts having widths (“diameters”) of 300 nm at their distal ends on an agar plate (top) and an image of the agar plate after overnight culture (bottom). As shown in this figure, the 300 nm post sample surrounded by a flat border region has a very distinct border region of dramatic bacterial growth, but no growth at all occurs at a center region where 300 nm posts are located. FIG. 15B depicts an image of a substrate bearing raised structures of posts having widths (“diameters”) of 1.5 μm at their distal ends (top) and an image of the agar plate after overnight culture (bottom). This figure shows that the 1.5 μm post sample shows a very distinct ‘border’ region for a room temperature agar plate; colonies very closely matched the linear edge of this pattern, indicating that the non-wetting region remained sterile and did not allow any bacteria to contaminate the agar. FIG. 15C depicts an image of a substrate bearing raised structures of posts having widths (“diameters”) of 5 μm at their distal ends (top) and an image of the agar plate after overnight culture with the substrate (bottom). In this figure, there is very clearly a significant growth of colonies in the border of flat, unetched Si around the etched, patterned region, indicating that there was much more contamination of the flat, wetting region than the patterned, non-wetting region. However, there are a few small colonies from the non-wetting region evident in the agar plate.

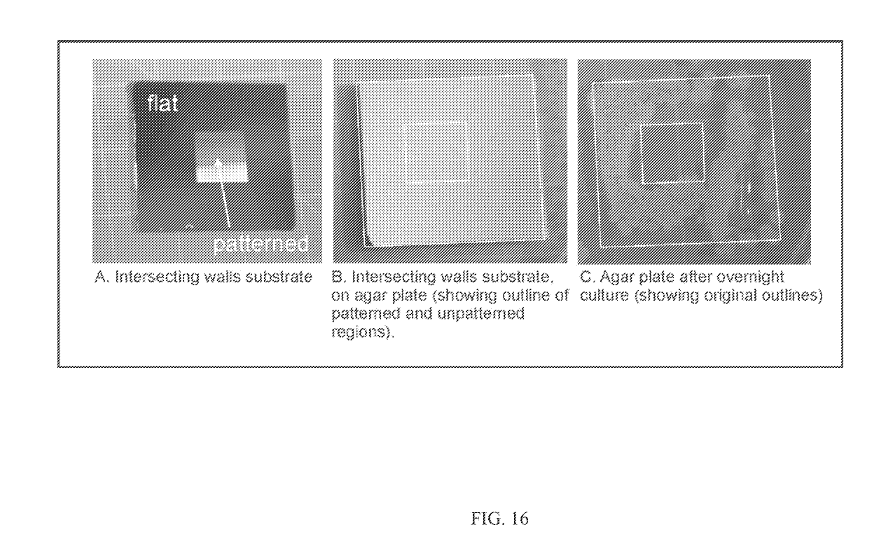

FIG. 16 shows that closed-cell structures with width of the walls of 1.3 microns remain sterile upon splashing of contaminated liquid as well. Here again, an area corresponding to the patterned surface is free of microorganisms, while the area corresponding to the flat surface has significant microbial growth.

These results suggest that bacterial attachment in energetic exclusion experiments under dynamic conditions (such as splashing, pouring, or sprinkling of contaminated liquid) is a function of the feature size of the superhydrophobic surface structure. It was found that only the posts of 1.5 μm and 300 nm appeared to cause a complete lack of bacterial attachment, meaning that the 5 μm diameter posts were large enough in area for some (small) degree of surface attachment. Therefore, to be sterile after splashing of contaminated liquid, the superhydrophobic surfaces should have posts with widths that are smaller than the bacteria themselves, i.e., less than about 2 microns for the case of P. aeruginosa. In situ observation of bacterial swimming at the air-liquid interface of a non-wetting droplet (using a water-based immersion lens, and phase contrast imaging) also confirmed, to some extent, that sporadic bacterial attachment to posts occurred for the 5 μm posts, with no attachment at all for the 300 nm and 1.5 μm posts. The absolute feature size (i.e. post diameter) is an important parameter in the control of bacterial attachment to superhydrophobic surfaces, and not simply the solid area fraction traditionally used to characterize superhydrophobic surfaces. Therefore, the presence of superhydrophobicity alone and the ability of the droplets to withdraw from the surface are not sufficient to ensure the absence of bacterial adhesion upon contact.

### Example 4

### Growth of Bacillus Subtilis on Raised Post Structures by Physical Exclusion

B. subtilis was also grown on an array of raised structures spaced at dimensions less than the longest dimension of the B. subtilis cell. A Si substrate comprising of 300 nm diameter posts, with a pitch of 0.9 μm, was immersed in B. subtilis (JH642 strain) culture containing MSgg growth medium, for a period of 12 h at room temperature, then rinsed with PBS. As shown by SEM imaging in FIG. 7B, the cells were found to only pack on the tips, and are isolated from each other. With limited surface contact, and with a large accessible porous volume underneath, these cells can be more easily removed than from a flat surface, either by mechanical or chemical methods, or a combination thereof.

### Example 5

### Growth of E. coli on Raised Structures by Physical Exclusion

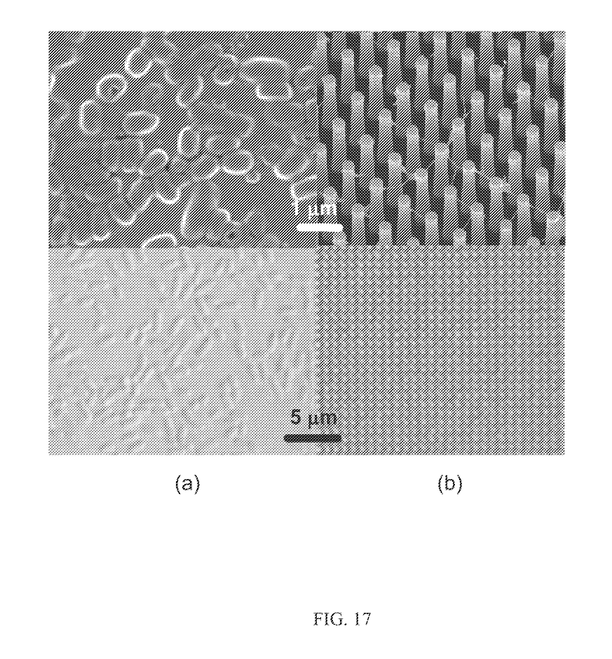

The arrangement of E. coli grown on arrays of raised posts was studied. A Si substrate comprising 300 nm diameter posts, with a pitch of 0.9 μm, was immersed in an E. coli (ZK2686 strain) culture containing TB growth medium, for a period of 12 h at room temperature, then rinsed with PBS. As shown in FIG. 17 (right images), when both the spacing between posts and the widths of the posts were less than the smallest dimension of the E. coli, no E. coli cells were found to remain on the tops of the posts after rinsing. The bacteria grown on these structures have reduced adhesion and have much easier detachment/removal than from a flat surface where many cells could be observed (left images).

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-39. (canceled)

40. An article having an antibiotic surface, said surface comprising:
a substrate; and
a plurality of raised structures on a face of said substrate, said raised structures defined by interstructure spacings, widths at their basal ends, and widths at their distal ends; wherein
said distal widths are less than about 50 μm, wherein said distal widths are selected to be less than five times the largest dimension of a microorganism in a contaminated liquid source that contacts the article, and
said surface is superhydrophobic.

41. An article having an antibiotic surface, said surface comprising:
a substrate; and
a plurality of raised structures on a face of said substrate, said raised structures defined by interstructure spacings, widths at their basal ends, and widths at their distal ends; wherein
said distal widths are less than about 50 μm, wherein said distal widths are selected to be less than five times the largest dimension of a microorganism in a contaminated liquid source that contacts the article;
said interstructure spacings are less than about 5 μm; and
said interstructure spacings are selected to be less than the largest dimension of said microorganism.

42. The article of claim 40, wherein said distal widths are less than about 20 μm or less than about 5 μm.

43. The article of claim 40, wherein said surface has a contact angle in the range of about 140° to about 180°.

44. The article of claim 40, wherein said basal widths are greater than said distal widths by a factor greater than about 1:1, greater than about 2:1 or greater than about 10:1.

45. The article of claim 40, wherein said distal widths are selected to be less than about three times the largest dimension of a microorganism in a contaminated liquid source that contacts the article.

46. The article of claim 40, wherein said raised structures are posts having shapes selected from circular, elliptical, polygonal, S-shaped in cross-section and cylindrical, conical, pyramidal, random, or combinations thereof.

47. The article of claim 46, wherein said posts are branched T-shaped, Y-shaped, X-shaped, or I-shaped in cross-section.

48. The article of claim 40, wherein said raised structures define channels, grooves, or closed-cell structures.

49. The article of claim 48, wherein said raised structures define closed-cell structures and said closed-cell structures are honeycombs or bricks.

50. The article of claim 48, wherein said channels, grooves or closed-cell structures are round-bottomed.

51. The article claim 40, wherein
said interstructure spacings are less than about 5 μm; and
said interstructure spacings are selected to be less than the largest dimension of said microorganism.

52. The article of claim 41, wherein said surface is superhydrophobic or is not superhydrophobic.

53. The article of claim 40, wherein the article is a medical device.

54. A method of inhibiting the attachment of a microorganism to a substrate, the method comprising:
transiently contacting the article of claim 40 with a contaminated liquid, thereby inhibiting the attachment of said microorganism to said substrate.

55. The method of claim 54, wherein said microorganism is an aspected microorganism having a length and a transverse diameter, and wherein said distal widths are less than about the transverse diameter of said microorganism.

56. The method of claim 54, wherein said distal widths are less than about 1 μm.

57. The method of claim 54, wherein said microorganism is a bacterium, virus or fungus, and the surfaces reduce the attachment of said microorganism during energetic exclusion under dynamic conditions when said distal widths are smaller than about 3-5 times the size of the bacterial, fungal, or viral cell or wherein the surfaces remain fully sterile during energetic exclusion under dynamic conditions where said distal widths of the features are less than the size of the microorganism.

58. The method of claim 54, wherein said microorganism is a bacterium, virus or fungus and the surfaces physically exclude said microorganism under static conditions when the interstructure spacing is smaller than the smallest dimension of said microorganism and the microorganism then attaches at the tips of said raised structures with limited contact.

59. The method of claim 54, wherein said microorganism is a bacterium, virus or fungus and the surfaces both energetically and physically exclude said microorganism under either dynamic or static exposures when both the width and the interstructure distance are smaller than the smallest dimension of the microorganism.
